(19) United States
(12) Patent Application Publication
(10) Pub. No.: US 2021/0214734 A1
(43) Pub. Date: Jul. 15, 2021

(54) METHODS FOR SELECTIVELY INHIBITING MOLECULAR CHAPERONE CLIENTS AND COMPOSITIONS FOR USE THEREOF

(71) Applicant: Research Foundation for the State University of New York, Albany, NY (US)

(72) Inventors: Mehdi Mollapour, Fayetteville, NY (US); Giorgio Colombo, Pavia (IT); Mark Woodford, Baldwinsville, NY (US); Gennady Bratslavsky, Fayetteville, NY (US); Dimitra Bourboulia, Fayetteville, NY (US)

(21) Appl. No.: 17/099,692

(22) Filed: Nov. 16, 2020

Related U.S. Application Data
(60) Provisional application No. 62/960,757, filed on Jan. 14, 2020.

(51) Int. Cl. C12N 15/70; C12N 15/62; C12P 21/02; C07K 14/47

(52) U.S. Cl. CPC C12N 15/70; C07K 14/4702; C12P 21/02; C12N 15/62

(57) ABSTRACT

The present disclosure relates to a method of identifying an agent-of-interest that alters binding or activity of a client protein to a chaperone, co-chaperone, or chaperone-co-chaperone complex, the method including: determining a three-dimensional (3D) structure of a client protein-of-interest; evaluating the 3D structure of the client protein-of-interest to identify an unstable substructure of the 3D structure of the client protein-of-interest; and determining an amino acid sequence of the unstable substructure of the 3D structure of the client protein-of-interest to identify an agent-of-interest that alters binding or activity of a client protein to a chaperone, co-chaperone, or chaperone-co-chaperone complex.

### CROSS REFERENCE TO RELATED APPLICATIONS

This application claims the benefit of the filing date of U.S. Provisional Application No. 62/960,757, filed Jan. 14, 2020. The content of this earlier filed application is hereby incorporated by reference herein in its entirety.

### GOVERNMENT INTERESTS

This invention was made with government support under grant no. GM124256 awarded by National Institutes of Health. The government has certain rights in the invention.

### INCORPORATION OF THE SEQUENCE LISTING

The present application contains a sequence listing the content of which is hereby incorporated by reference.

### FIELD OF THE INVENTION

The present disclosure relates to the field of client protein, chaperone, co-chaperone, and chaperone-co-chaperone complex interactions. For example, the present disclosure relates to selectively inhibiting a preselected client protein, such as an HSP90 client protein, by identifying and synthesizing one or more agents-of-interest that alters binding or activity of a client protein to a chaperone, co-chaperone, or chaperone-co-chaperone complex and use thereof.

### BACKGROUND

Chaperone proteins play important regulator roles in the cell, affecting numerous biological processes by inducing changes in related client proteins. For example, the Hsp90 family of molecular chaperones play key roles in cell proteostasis by balancing the folding, activation, and turnover of a diverse set of client proteins, many of which are fundamental for cancer development. Hsp90 functions depend on ATP hydrolysis and interactions with clients and co-chaperones. Hsp90 inhibition by ATP competitive inhibitors, however, problematically leads to the indiscriminate depletion of all Hsp90 clients, thereby causing the upregulation of the heat shock response which ultimately protects cancer cells from apoptosis and causes toxicity.

The formation of client:Hsp90 complexes is a critical step in the regulation of specific client activities. Specificity in the selection of clients that lack sequence and structural homology (See e.g., Taipale et al., Nat Rev Mol Cell Biol. 2010, 11, 515-528; or Taipale et al., Cell 2012, 150, 987-1001) is acquired through recruiter cochaperones that provide the essential recognition/discrimination elements (See Röhl et al., Trends Biochem. Sci. 2013, 38, 253-262). In this framework, Hsp90, its co-chaperones and the clients engage in multicomponent assemblies, stabilized by dynamic protein-protein interactions (PPIs). Co-chaperones such as Cdc37 control the entry of kinases and other clients into the chaperone cycle (See e.g., Caplan et al., Trends. Cell. Biol. 2007, 17, 87-92, Karnitz et al., Sci. STKE. 2007, 2007:pe2022, and Keramisanou et al., Molecular Cell 2016, 62, 260-27), while other co-chaperones, such as Aha1, provide additional layers of regulation by modulating the rates of ATP hydrolysis (See Zuehlke et al., BIOPOLYMERS 2010, 93 211-217). No specific structural elements or surface characteristics have been proposed as Hsp90-binding determinants (See e.g., Citri et al., J. Biol. Chem. 2006, 281, 14361-14369; Prince et al., J. Biol. Chem. 2004, 279, 39975-39981, Scroggins et al., Biochemistry 2003, 42, 12550-12561 and Xu et al., Molecular Cell 2012, 47, 434-443. Critical to Hsp90 mechanisms is that the interactions involved are conformationally heterogeneous, short-lived and relatively weak, with different clients interacting in distinct ways (See e.g. Pricer et al., Accounts of chemical research, 2017, 50, 584-589).

The inventors have found that these observations unveil a new opportunity for the design of client-selective chemical tools based on the idea that perturbing the (weak) interactions with (any of) the members of the chaperone assembly can impair a client's folding. The lack of consensus binding motifs suggests the possibility to target unique interaction surfaces in order to specifically disrupt key client:chaperone or client:cochaperone interactions.

The inventors have observed that general inhibitors of chaperone proteins, such as Hsp90, directly bind to Hsp90 and inhibit its chaperone activity problematically inhibiting the function of many client proteins. The resulting toxicity precludes the use of general inhibitors and made it difficult to identify chemicals such as drug candidates that selectively inhibit chaperone protein-mediated effects on client proteins.

What is needed are methods of identifying and making selective inhibitors that may act upon a specific or preselected client protein and its related chaperone, co-chaperone, or chaperone-co-chaperone complexes without detrimentally impacting all protein clients.

### SUMMARY

Other features and advantages of the present compositions and methods are illustrated in the description below, the drawings, and the claims.

In some embodiments, the present disclosure relates to a method of identifying an agent-of-interest that alters binding or activity of a client protein to a chaperone, co-chaperone, or chaperone-co-chaperone complex, the method including: determining a three-dimensional (3D) structure of a client protein-of-interest; evaluating the 3D structure of the client protein-of-interest to identify an unstable substructure of the 3D structure of the client protein-of-interest; and determining an amino acid sequence of the unstable substructure of the 3D structure of the client protein-of-interest to identify an agent-of-interest that alters binding or activity of a client protein to a chaperone, co-chaperone, or chaperone-co-chaperone complex.

In some embodiments, the present disclosure relates to a method of inhibiting, treating, or preventing cancer or metastatic cancer in a subject, the method including, administering a therapeutically effective amount of an agent-of-interest identified by a method of the present disclosure, or a pharmaceutically acceptable salt or a derivative thereof, to a subject in need of treatment.

In some embodiments, the present disclosure relates to a method of identifying an agent-of-interest that alters binding or activity of an Hsp90 client protein to an Hsp90 chaperone, Hsp90 co-chaperone, or Hsp90 chaperone-co-chaperone complex, the method including: evaluating a 3D structure of an Hsp90 client protein-of-interest to identify an unstable substructure of the 3D structure of the Hsp90 client protein-of-interest; determining an amino acid sequence of the unstable substructure of the 3D structure of the Hsp90 client protein-of-interest to identify an Hsp90 agent-of-interest that alters binding or activity of an Hsp90 client protein to an Hsp90 chaperone, Hsp90 co-chaperone, or Hsp90 chaperone-co-chaperone complex.

In some embodiments, the present disclosure relates to a synthetic selective peptide inhibitor, including: an amino acid sequence having at least 90% sequence identity to SEQ ID NO: 1, 2, 3, 4, 5, 6, 7, 8, 9, or 10.

In some embodiments, the present disclosure relates to one or more synthetic peptides, including: an amino acid sequence having at least 90% sequence identity to SEQ ID NO: 1, 2, 3, 4, 5, 6, 7, 8, 9, or 10, wherein the one or more synthetic peptides alter binding or activity of an Hsp90 client protein to an Hsp90 chaperone, Hsp90 co-chaperone, or Hsp90 chaperone-co-chaperone complex.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. These and other features of this disclosure will be more readily understood from the following detailed description of the various aspects of the disclosure taken in conjunction with the accompanying drawings that depict various embodiments of the disclosure, in which:

FIG. 1 depicts 3D structures and the names of several proteins analyzed in accordance with the present disclosure, wherein the regions colored in red are the substructures predicted to undergo unfolding and to be points of interaction with the members of the Hsp90 chaperone machinery.

FIG. 2A depicts ¹⁹F NMR experiments showing the binding of AbI and Braf peptides to the proteins of the Hsp90 chaperone system. The subpanels indicate the interactions of: (line 1) peptide A01; (line 2) peptide A02; (line 3) peptide B-Raf01; (line 4) peptide B-Raf02; (line 5) wherein all peptides were mixed together and added to the solution containing the protein. The NMR signals show no variation compared to the situation with the single peptides, indicating that there is no competition among the different sequences. FIG. 2B depicts ¹⁹F NMR experiments showing the binding of GR peptides to the proteins of the Hsp90 chaperone system. (1) peptide GR-01; (2) GR-02; (3) both peptides are mixed together and added to the solution containing the protein. The NMR signals show that GR-02 binds to all the protein, but the binding effect is higher on CDC37. No significant difference are observed in the binding of GR-02 in presence of GR-01.

FIG. 3A depicts an immunoblot where HEK293 cells were treated with 10 μM A01 or A02 with and without TAT. After 36 hours, lysate was prepared from the cells followed by immunoprecipitation of endogenous Hsp90. Inputs and co-immunoprecipitated proteins were evaluated by immunoblotting as depicted herein. GAPDH was used as a loading control. NT=no treatment. FIG. 3B depicts an immunoblot where Hsp90α-FLAG or Hsp90β-FLAG were overexpressed in HEK293 cells. Hsp90 binding to biotinylated A01 and A02 was examined by immunoblotting as depicted herein.

FIGS. 4A-4G depict various immunoblots. FIG. 4A depicts endogenous Hsp90 immunopreciptated from HEK293 cells treated with the indicated amounts of B-Raf peptides. Hsp90 binding to B-raf was evaluated by immunoblotting. FIG. 4B depicts binding of Hsp90 and Cdk4 evaluated by immunoblotting after treatment with the indicated concentrations of Cdk4-01 and Cdk4-02 and immunoprecipitation of endogenous Hsp90. FIG. 4C depicts binding of Hsp90 and c-Src evaluated by immunoblotting after treatment with the indicated concentrations of c-Src-01 and c-Src-02 and immunoprecipitation of endogenous Hsp90. FIG. 4D depicts HEK293 cells treated with the indicated amounts of GR-01 or GR-02 then examined for total GR protein by immunoblotting. FIG. 4E depicts representative microscopy images of drug uptake in HEK293 cells treated with 10 μM FAM-labeled peptides for 24 h. Scale bar=100 μm. NT=non-treated. FIG. 4F depicts apoptosis in cancer cells evaluated by immunoblotting for the apoptotic marker cleaved caspase-3 in 786-O cells treated with the kinase peptide mimics. FIG. 4G depicts apoptosis in cancer cells evaluated by immunoblotting for the apoptotic marker cleaved caspase-3 in 786-O cells treated with the kinase peptide mimics.

FIGS. 5A-5D depict ¹⁹F NMR experiments to check the binding of AbI and Braf peptides to HSA. FIG. 5A refers to Peptide AblO1; FIG. 5B refers to AblO2; FIG. 5C refers to Braf01-pep; FIG. 5D refers to Braf02-pep. No peptide is depicts showing a significant difference in their ¹⁹F signals in the presence of protein, indicating that they do not interact with HSA.

FIG. 6 depicts ¹⁹F NMR experiments to check the purity of synthesized peptides.

FIGS. 7A-7D depict ¹⁹F NMR experiments to check the stability of synthesized peptides in TRIS buffer.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

The disclosed method and compositions may be understood more readily by reference to the following detailed description of particular embodiments and the Example included therein and to the Figures and their previous and following description.

The present disclosure relates to client protein, chaperone, co-chaperone, and chaperone-co-chaperone complex interactions. For example, the present disclosure relates to selectively inhibiting a preselected client protein, such as an HSP90 client protein, by identifying and synthesizing one or more agents-of-interest that alters binding or activity of a client protein to a chaperone, co-chaperone, or chaperone-co-chaperone complex and use thereof. In embodiments, methods of the present disclosure include a method of identifying an agent-of-interest that alters binding or activity of a client protein to a chaperone, co-chaperone, or chaperone-co-chaperone complex, the method including: determining a three-dimensional (3D) structure of a client protein-of-interest; evaluating the 3D structure of the client protein-of-interest to identify an unstable substructure of the 3D structure of the client protein-of-interest; determining an amino acid sequence of the unstable substructure of the 3D structure of the client protein-of-interest to identify an agent-of-interest that alters binding or activity of a client protein to a chaperone, co-chaperone, or chaperone-co-chaperone complex.

Advantages of the present disclosure include: predicting interaction interfaces of one or more different clients based on the structure of one or more isolated clients to design one or more selective peptide inhibitors of protein-protein interactions in chaperone complexes. In embodiments, the design method of the present disclosure is based on a computational method developed for the prediction of locally unstable substructures in proteins. Unstable substructures represent potential ideal points of interaction with the Hsp90 machinery (See e.g., K. A. Verba, D. A. Agard, Trends in biochemical sciences 2017, 42, 799-811; and K. A. Verba, R. Y. Wang, A. Arakawa, Y. Liu, M. Shirouzu, S. Yokoyama, D. A. Agard, Science 2016, 352, 1542-1547). This knowledge is translated into the development of peptides such as one or more therapeutic selective inhibitors spanning the predicted interaction sites to engage different constituents of a client, chaperone, co-chaperone, chaperone-co-chaperone complex such as the Hsp90 complex (Hsp90, Cdc37, Aha1). In embodiments, the therapeutic peptides such as selective inhibitors of the present disclosure are cell permeable and selectively interfered with the association of their respective clients (such as the Hsp90 chaperone machinery) ultimately causing apoptosis in cancer cells. In embodiments, an ab initio, physics-based characterization of protein stability is leveraged for the selective chemical targeting of chaperone:client interactions in multicomponent complexes. In embodiments, this is achieved without significant indiscriminate inhibition or degradation of all clients, and defining pharmacophoric requirements for the development of PPI targeting molecules with therapeutic potential.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein, the phrase “an agent of interest that alters binding or activity” can mean a compound that inhibits or stimulates or can act on another protein which can inhibit or stimulate the protein-protein interaction of a complex of two proteins.

As used herein, the term “client protein” refers to a protein that can be manipulated or processed, for example, folding by one or more chaperone proteins. Examples of client proteins include but are not limited to kinases.

As used herein, the term “chaperone complex” or “chaperone-co-chaperone complex” or “heterocomplex” refers to a group of two or more associated polypeptide chains or proteins. In an aspect, a chaperone-co-chaperone complex can refer to Hsp90α-Cdc37 or Hsp90b-Cdc37. Proteins or polypeptide chains (e.g., chaperone or chaperone protein) in a chaperone complex or chaperone-co-chaperone complex can be linked by non-covalent protein-protein interactions. A “chaperone” or “chaperone protein” are also known as “molecular chaperones”. A “chaperone” or “chaperone protein” or “molecular chaperone” is a protein that assists the covalent folding or unfolding and the assembly or disassembly of other macromolecular structures.

As used herein the “degree of identity” refers to the relatedness between two amino acid sequences or between two nucleotide sequences and is described by the parameter “identity”. In embodiments, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid or nucleotide in the two aligned sequences on a given position in the alignment; and 3) dividing the number of exact matches with the length of the reference sequence.

In one embodiment, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid; or nucleotide in the two aligned sequences on a given position in the alignment; and 3) dividing the number of exact matches with the length of the longest of the two sequences. In some embodiments, the degree of sequence identity refers to and may be calculated as described under “Degree of Identity” in U.S. Pat. No. 10,531,672 starting at Column 11, line 56. U.S. Pat. No. 10,531,672 is incorporated by reference in its entirety.

In embodiments, an alignment program suitable for calculating percent identity performs a global alignment program, which optimizes the alignment over the full-length of the sequences. In embodiments, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available on the world wide web at www.ebi.ac.uk/Tools/psa/. In some embodiments a global alignment program uses the Needleman-Wunsch algorithm and the sequence identity is calculated by identifying the number of exact matches identified by the program divided by the “alignment length”, where the alignment length is the length of the entire alignment including gaps and overhanging parts of the sequences.

As used herein the terms “drug,” “drug substance,” “active agent,” “active pharmaceutical ingredient,” and the like, refer to a compound that may be used for treating a subject in need of treatment. Non-limiting examples of such a compound include peptides such as one or more selective peptide inhibitors of the present disclosure.

As used herein the terms “drug product,” “pharmaceutical dosage form,” “dosage form,” “final dosage form” and the like, refer to a pharmaceutical composition that is administered to a subject in need of treatment and generally may be in the form of tablets, capsules, sachets containing powder or granules, liquid solutions or suspensions, patches, and the like.

As used herein the term “excipient” or “adjuvant” refers to any inert substance.

“Homologue” means an entity having a certain degree of identity or “homology” with the subject amino acid sequences and the subject nucleotide sequences.

A “homologous sequence” includes a polynucleotide or a polypeptide having a certain percent, e.g., 80%, 85%, 90%, 95%, or 99% of sequence identity with another sequence. Percent identity means that, when aligned, that percentage of bases or amino acid residues are the same when comparing the two sequences. Amino acid sequences are not identical, where an amino acid is substituted, deleted, or added compared to the subject sequence. The percent sequence identity typically is measured with respect to the mature sequence of the subject protein, i.e., following removal of a signal sequence, for example. Typically, homologues will include the same active site residues as the subject amino acid sequence. Homologues may also retain activity, although the homologue may have different properties than the wild-type.

The term “hydrate” describes a solvate including the drug substance and a stoichiometric or non-stoichiometric amount of water.

As used herein, “nucleotide sequence” or “nucleic acid sequence” refers to an oligonucleotide sequence or polynucleotide sequence and variants, homologues, fragments and derivatives thereof. The nucleotide sequence may be of genomic, synthetic or recombinant origin and may be double-stranded or single-stranded, whether representing the sense or anti-sense strand. As used herein, the term “nucleotide sequence” includes genomic DNA, cDNA, synthetic DNA, and RNA.

As used herein the term “pharmaceutically acceptable” substances refers to those substances which are within the scope of sound medical judgment suitable for use in contact with the tissues of subjects without undue toxicity, irritation, allergic response, and the like, and effective for their intended use.

As used herein the term “pharmaceutically acceptable vehicle” refers to a diluent, adjuvant, excipient or carrier with which a compound is administered.

As used herein the term “pharmaceutical composition” refers to the combination of one or more drug substances and one or more excipients such as one or more selective peptide inhibitors of the present disclosure and one or more pharmaceutically acceptable vehicles with which the one or more selective peptide inhibitors is administered to a subject.

As used herein, the term “pharmaceutically acceptable salt” refers to a salt of a compound, which possesses the desired pharmacological activity of the parent compound. Non-limiting examples of pharmaceutically acceptable salts include: acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, for example, an alkali metal ion, an alkaline earth ion, or an aluminum ion; or coordinates with an organic base such as ethanolamine, diethanolamine, triethanolamine, N-methylglucamine, and the like.

As used herein, “polypeptide” is used interchangeably with the terms “amino acid sequence”, “peptide” and/or “protein”.

As used herein, the terms “polypeptide sequence” and “amino acid sequence” are used interchangeably.

As used herein the term “prevent”, “preventing” and “prevention” of cancer means (1) reducing the risk of a patient who is not experiencing symptoms of cancer from developing cancer, or (2) reducing the frequency of, the severity of, or a complete elimination of cancer in a subject.

As used herein, the term “sequence” can either be referring to a polypeptide sequence or a nucleic acid sequence, depending of the context.

As used herein the term “subject” includes humans, animals or mammals. The terms “subject” and “patient” may be used interchangeably herein.

As used herein the term “therapeutically effective amount” means the amount of a compound that, when administered to a subject for treating or preventing cancer, is sufficient to have an effect on such treatment or prevention of the cancer. A “therapeutically effective amount” can vary depending, for example, on the compound, the severity of the cancer, the etiology of the cancer, comorbidities of the subject, the age of the subject to be treated and/or the weight of the subject to be treated. A “therapeutically effective amount” is an amount sufficient to alter the subjects' natural state.

As used herein the term “solvate” describes a molecular complex including the drug substance (e.g., selective peptide inhibitor) and a stoichiometric or non-stoichiometric amount of one or more pharmaceutically acceptable solvent molecules.

As used herein the term “treat”, “treating” and “treatment” of cancer means reducing the frequency of symptoms of cancer, eliminating the symptoms of cancer, avoiding or arresting the development of cancer, ameliorating or curing an existing or undesirable symptom caused by cancer, and/or reducing the severity of symptoms of cancer.

### DETAILED DESCRIPTION OF CERTAIN EMBODIMENTS

Disclosed herein are compositions and methods that serve to overcome the problem of identifying drug candidates that selectively inhibit chaperone protein-mediated effects on client proteins. Embodiments further include methods and assays that design active agent candidates for selective inhibition of client proteins. Further, the disclosed assays and methods can be used to design drug candidates that affect how chaperone proteins affect client proteins. As such, the methods and assays disclosed herein can be used to design large sets of new chemicals for their ability to affect specific individual, or sets of, client proteins.

In embodiments, the present disclosure relates to methods for identifying an agent-of-interest that alters binding or activity of a client protein to a chaperone, co-chaperone, or chaperone-co-chaperone complex, the method including: determining a three-dimensional (3D) structure of a client protein-of-interest; evaluating the 3D structure of the client protein-of-interest to identify an unstable substructure of the 3D structure of the client protein-of-interest; and determining an amino acid sequence of the unstable substructure of the 3D structure of the client protein-of-interest to identify an agent-of-interest that alters binding or activity of a client protein to a chaperone, co-chaperone, or chaperone-co-chaperone complex.

In embodiments, the present disclosure relates to methods for identifying an agent-of-interest that alters binding or activity of an Hsp90 client protein to an Hsp90 chaperone, Hsp90 co-chaperone, or Hsp90 chaperone-co-chaperone complex, the method including: evaluating a 3D structure of an Hsp90 client protein-of-interest to identify an unstable substructure of the 3D structure of the Hsp90 client protein-of-interest; and determining an amino acid sequence of the unstable substructure of the 3D structure of the Hsp90 client protein-of-interest to identify an Hsp90 agent-of-interest that alters binding or activity of an Hsp90 client protein to an Hsp90 chaperone, Hsp90 co-chaperone, or Hsp90 chaperone-co-chaperone complex.

In some embodiments, the altered activity can be the activity of the client protein to the chaperone complex or chaperone-co-chaperone complex. In embodiments, the activity can be kinase activity, phosphatase activity, ligase activity, E3 ligase activity or transcription factor activity or a combination thereof.

In some embodiments, non-limiting examples of agents-of-interest include, but are not limited to, small molecules, biological agents, peptides, polypeptides, chemical compounds and the like. In some embodiments, the one or more agents-of-interest as identified may alter cancer cell invasion and motility. In an aspect, the one or more agents-of-interest can reduce or inhibit cancer cell invasion. In an aspect, the one or more agents-of-interest can reduce or inhibit cancer cell motility. In a further aspect, the one or more agents of interest can alter the phosphorylation state of a chaperone protein or co-chaperone protein.

In some embodiments, one or more agents-of-interest include one or more polypeptides comprising or consisting of the amino acid sequence of SEQ ID NO:1, SEQ ID NO 2: SEQ ID NO:3, SEQ ID NO:4, SEQ ID NO 5: SEQ ID NO:6, SEQ ID NO:7, SEQ ID NO 8: SEQ ID NO:9, and/or SEQ ID NO: 10. In some embodiments, one or more agents-of-interest include one or more polypeptides comprising or consisting of an amino acid sequence having at least 90%, at least 95%, at least 97%, or at least 99% sequence identity to SEQ ID NO:1, SEQ ID NO 2: SEQ ID NO:3, SEQ ID NO:4, SEQ ID NO 5: SEQ ID NO:6, SEQ ID NO:7, SEQ ID NO 8: SEQ ID NO:9, or SEQ ID NO: 10. In some embodiments, the amino acid sequences may include one or more conservative substitutions, such that the upon alteration of one or more amino acids, the functionality of the amino acid sequence is not changed. In some embodiments, the one or more agents-of-interest include an amino acid that acts to stabilize the one or more agents-of-interest. In embodiments, any peptide protecting group can be included within the amino acid sequence of the agent-of-interest. Non-limiting examples of a protective group may include 4-Fluoro-L-phenylalanine as shown in Table 1. In some embodiments, the agent-of-interest includes the amino acid sequence of SEQ ID. NOS: 1-10, without the 4-Fluoro-L-phenylalanine protecting group.

In some embodiments, the present disclosure provides a synthetic selective peptide inhibitor, including: an amino acid sequence having at least 90% sequence identity to SEQ ID NO: 1, 2, 3, 4, 5, 6, 7, 8, 9, or 10. In some embodiments, the present disclosure provides one or more agents-of-interest including an amino acid sequence having at least 90% sequence identity to SEQ ID NO: 1, 2, 3, 4, 5, 6, 7, 8, 9, or 10, wherein the agent-of-interest alters binding or activity of a client protein to a chaperone, co-chaperone, or chaperone-co-chaperone complex. In some embodiments, the present disclosure provides one or more agents-of-interest including an amino acid sequence having at least 90% sequence identity to SEQ ID NO: 1, 2, 3, 4, 5, 6, 7, 8, 9, or 10, wherein the agent-of-interest alters binding or activity of an Hsp90 client protein to an Hsp90 chaperone, Hsp90 co-chaperone, or Hsp90 chaperone-co-chaperone complex. In some embodiments, the present disclosure provides one or more agents-of-interest including an amino acid sequence having at least 95%, at least 97%, or at least 99% sequence identity to SEQ ID NO: 1, 2, 3, 4, 5, 6, 7, 8, 9, or 10, wherein the agent-of-interest alters binding or activity of an Hsp90 client protein to an Hsp90 chaperone, Hsp90 co-chaperone, or Hsp90 chaperone-co-chaperone complex. In some embodiments, the present disclosure provides one or more agents-of-interest consisting of the amino acid sequences of SEQ ID NO: 1, 2, 3, 4, 5, 6, 7, 8, 9, or 10, wherein the agent-of-interest alters binding or activity of an Hsp90 client protein to an Hsp90 chaperone, Hsp90 co-chaperone, or Hsp90 chaperone-co-chaperone complex.

In some aspects, the methods may include assaying one or more agents-of-interest for altering phosphorylation of HSP90, and identifying and selecting one or more agents-of-interest altering phosphorylation of HSP90. In an aspect, the method can further include assaying one or more agents of interest identified for altering phosphorylation of any chaperone, co-chaperone or client protein.

In an aspect, the method can further include assaying one or more agents-of-interest for altering post-translational modification of any chaperone, co-chaperone or client protein. In an aspect, the post-translation modifications can be selected from the group consisting of phosphorylation, acetylation, nitrosylation, methylation, ubiquitination, SUMOylation, acylation, O-GlcNAcylation, AMPylation and oxidation.

In embodiments, Hsp90 (heat shock protein 90) is a chaperone protein, known to play an important role in stabilizing proteins for normal cellular growth. Tumors are addicted to Hsp90 because they need Hsp90 for stability and activity of dysregulated oncoproteins and drivers of tumorigenesis. The term “90” refers that the molecular weight of Hsp90 is about 90 kDa. Hsp90 is expressed in all eukaryotes, such as yeast and mammals, including rodents (e.g., a mouse, a rat, etc.), primates (a human, a monkey, etc.), and the like. The following are examples of known Hsp90 sequences: yeast Hps90 (e.g., NCBI Accession No. NP_013911.1, NP_015084.1, etc.), human Hsp90 (e.g., NCBI Accession No. NP_001017963.2, NP_005339.3, etc.), mouse Hsp90 (e.g., NCBI Accession No. NP_034610.1, NP_032328.2, etc.), rat Hsp90 (e.g., NCBI Accession No. NP_786937.1, AAT99569.1, etc.), and the like. Hsp90 coding gene (e.g., mRNA) may be at least one selected from the group consisting of yeast Hsp90 (e.g., NCBI Accession No. NM_001182692.1, NM_001184054.1, etc.), human Hsp90 (e.g., NCBI Accession No. NM 001017963.2, NM_005348.3, etc.), mouse Hsp90 (e.g., NCBI Accession No. NM_010480.5, NM_008302.3, etc.), rat Hsp90 (e.g., NCBI Accession No. NM_175761.2, AY695393.1, etc.), and the like. (See for example, U.S. Pat. No. 9,956,244 (herein entirely incorporated by reference).

In some embodiments, chaperone proteins may include, but are not limited to, HsplOO, HspKM, Hspl lO, Hsp90a, Hsp90b, Grp94, Grp78, Hsp72, Hsp7I, Hsp70, Hsx70, Hsp60, Hsp47, Hsp40, Hsp27, Hsp20, hspbI2, HsplO, hspb7, Hspb6, Hspb4, HspBI, and alpha B crystallin.

In some embodiments, co-chaperone proteins may include, but are not limited to, Cdc37/p50, Ahal, auxilin, BAG1, CAIR-I/Bag-3, Chpl, Cyp40, Djpl, DnaJ, E3/E4-ubiquitin ligase, FKBP52, GAK, GroES, Hchl, Hip (Hsc70-interacting protein)/STI3, Hop (Hsp70/Hsp90 organizing protein)/STIPI, Mq, PPS, Sacsin, SGT, Snll, SODD/Bag-4, Swa2/Auxl, Tom34, Tom70, UNC-45, and WISp39.

In some embodiments, chaperone-co-chaperone complexes may include Hsp90b-Cdc37. Additionally, examples include a chaperone-co-chaperone grouping including any of the chaperone protein of HsplOO, Hspl04, Hspl lO, Hsp90a, Hsp90b, Grp94, Grp78, Hsp72, Hsp7I, Hsp70, Hsx70, Hsp60, Hsp47, Hsp40, Hsp27, Hsp20, hspbI2, HsplO, hspb7, Hspb6, Hspb4, HspBI, and alpha B crystallin.

In an aspect, the client protein may include kinases, phosphatases, ligases, E3 ligases and transcription factors. In an aspect, the client protein can be a polypeptide. In an aspect, the polypeptide can participate in cell motility, cytotoxicity, metastasis, survival, organ destruction, phosphorylation of HSP90beta, covalent modifications of chaperone proteins and/or a co-chaperone. Examples of client proteins include, but are not limited to, MAP3K15, RJPK1, RAF1, NTRK1, MAP3K6, GSG2, RIPK2, NEK2, PRKCB1, LIMK1, TGFBR1, LOC340371, PRKACG, CAMK28, 0081461, SGK3, NLK, and a fragment or derivative thereof. Additional examples include, but are not limited to, the following client proteins as shown in International Patent Publication No. WO 2019/157150 A1 (herein incorporated by reference in its entirety).

In some embodiments the present disclosure relates to a method of identifying an agent-of-interest that alters binding or activity of a client protein to a chaperone, co-chaperone, or chaperone-co-chaperone complex, the method including: determining a three-dimensional (3D) structure of a client protein-of-interest; evaluating the 3D structure of the client protein-of-interest to identify an unstable substructure of the 3D structure of the client protein-of-interest; and determining an amino acid sequence of the unstable substructure of the 3D structure of the client protein-of-interest to identify an agent-of-interest that alters binding or activity of a client protein to a chaperone, co-chaperone, or chaperone-co-chaperone complex. In some embodiments, the method may further include synthesizing a selective peptide inhibitor or agent-of-interest having an amino acid sequence having at least 90%, 95%, 97%, or 99% sequence identity to the amino acid sequence of the unstable substructure of the 3D structure of the client protein-of-interest. In some embodiments, the selective peptide inhibitor or agent-of-interest blocks protein interactions or alters binding or activity of a client protein to a chaperone, co-chaperone, or chaperone-co-chaperone complex, and/or induces apoptosis in cancer cells.

In some embodiments, evaluating the 3D structure of the client protein-of-interest to identify an unstable substructure of the 3D structure of the client protein-of-interest further includes predicting the unstable substructure of the 3D structure of the client protein-of-interest based on energy decomposition. In some embodiments, the client protein includes one or more proteins characterized as a heat shock protein (HSP), one or more Hsp90 client proteins, one or more proteins selected from a class including a steroid hormone, a receptor, a kinase, a non-signal transduction, a telomerase, or a CFTR, and/or one or more of c-Abl, c-Src, Cdk4, B-Raf or glucocorticoid receptor. In some embodiments, the chaperone is an Hsp90 chaperone. In some embodiments, the co-chaperone is an Hsp90 co-chaperone such as one of CDC37, or Aha1. In some embodiments, the chaperone-co-chaperone complex is an Hsp90 chaperone-co-chaperone complex. In some embodiments, the unstable substructure of the 3D structure of the client protein-of-interest is further characterized as an epitope. In some embodiments, the activity of the client protein to the chaperone-co-chaperone complex is kinase activity, E3 ligase activity, transcription factor activity, or a combination thereof. In some embodiments, the one or more agents-of-interest alter, inhibit, decrease, or destroy one or more cancer cells. In embodiments, the one or more agents-of-interest are characterized as permeable to a cell membrane. In some embodiments, the methods include assaying one or more agents-of-interest for altering post-translational medication of any chaperone, co-chaperone, or client protein.

In some embodiments, the present disclosure relates to a method of identifying an agent-of-interest that alters binding or activity of an Hsp90 client protein to an Hsp90 chaperone, Hsp90 co-chaperone, or Hsp90 chaperone-co-chaperone complex, the method including: evaluating a 3D structure of an Hsp90 client protein-of-interest to identify an unstable substructure of the 3D structure of the Hsp90 client protein-of-interest; and determining an amino acid sequence of the unstable substructure of the 3D structure of the Hsp90 client protein-of-interest to identify an Hsp90 agent-of-interest that alters binding or activity of an Hsp90 client protein to an Hsp90 chaperone, Hsp90 co-chaperone, or Hsp90 chaperone-co-chaperone complex.

### Methods of Treatment

Also disclosed herein are methods of inhibiting, preventing or treating cancer or metastatic cancer in a subject. In an aspect, the method can include administering to the subject a therapeutically effective amount of an agent-of-interest identified by any of methods disclosed herein or a salt or a derivative thereof, thereby inhibiting, preventing or treating cancer or metastatic cancer in the subject.

Disclosed herein are methods of treating cancer or metastatic cancer in a subject. In an aspect, the method can include identifying a subject in need of treatment; and administering a therapeutically effective amount of the agent-of-interest identified by the method disclosed herein or a salt or a derivative thereof.

Disclosed herein are methods of inhibiting cancer or metastatic cancer in a subject. In an aspect, the method can include: identifying a subject in need of treatment; and administering a therapeutically effective amount of the agent-of-interest identified by the method disclosed herein or a salt or a derivative thereof.

Disclosed herein are methods of preventing cancer or metastatic cancer in a subject. In an aspect, the method can include: identifying a subject in need of treatment; and administering a therapeutically effective amount of an agent-of-interest identified by the method disclosed herein or a salt or a derivative thereof.

The compositions described herein can be formulated to include a therapeutically effective amount of any of the agents-of-interest identified using any of the methods disclosed herein described herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of cancer.

The compositions described herein can be formulated in a variety of combinations. The particular combination of one or more of the agents-of-interest identified in any of the methods disclosed herein can vary according to many factors, for example, the particular the type and severity of the cancer.

The compositions described herein can be administered to the subject (e.g., a human patient) in an amount sufficient to delay, reduce, or preferably prevent the onset of clinical disease. Accordingly, in some aspects, the subject can be a human subject. In therapeutic applications, compositions are administered to a subject (e.g., a human patient) already with or diagnosed with cancer in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the cancer is delayed, hindered, or prevented, or the cancer or a symptom of the cancer is ameliorated. One or more of the symptoms can be less severe. Recovery can be accelerated in an individual who has been treated.

In some aspects, the cancer can be a primary or secondary tumor. In an aspect, the cancer can be a metastatic tumor. In other aspects, the primary or secondary tumor is within the patient's breast, lung, lung, prostate, head or neck, brain, bone, blood, colon, gastrointestinal track, esophagus or liver. In yet other aspects, the cancer has metastasized. In some aspects, the cancer may metastasize to one or more of the following sites: the breast, lung, liver or bone.

Disclosed herein, are methods of treating a patient with cancer. The cancer can be any cancer. In some aspects, the cancer can be breast cancer, lung cancer, brain cancer, liver cancer, prostate cancer, head or neck cancer, a blood cancer, colon cancer, gastrointestinal track cancer, bone cancer or esophageal cancer. In an aspect, the subject has been diagnosed with cancer prior to the administering step.

The therapeutically effective amount or dosage of the any of the agents-of-interest identified in any of the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the cancer symptoms), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of the compositions as disclosed herein can be administered to a subject as a single dose, either as a bolus or by infusion over a relatively short period of time, or can be administered using a fractionated treatment protocol in which multiple doses are administered over a more prolonged period of time.

Alternatively, continuous intravenous infusions sufficient to maintain therapeutically effective concentrations in the blood are also within the scope of the present disclosure.

The compositions described herein can be administered in conjunction with other therapeutic modalities to a subject in need of therapy. The present compounds can be given to prior to, simultaneously with or after treatment with other agents or regimes.

In some embodiments, the present disclosure relates to a method of inhibiting, treating, or preventing cancer or metastatic cancer in a subject, the method including, administering a therapeutically effective amount of an agent-of-interest identified by the method of the present disclosure or a pharmaceutically acceptable salt or a derivative thereof, to a subject in need of treatment.

### Pharmaceutical Compositions

As disclosed herein, are pharmaceutical compositions, including one or more of the therapeutic compositions disclosed herein.

In some embodiments, pharmaceutical compositions may include one or more agents-of-interest such as a synthetic selective peptide inhibitor, including: an amino acid sequence having at least 90% sequence identity to SEQ ID NOS: 1, 2, 3, 4, 5, 6, 7, 8, 9, or 10. In some embodiments, the active agents for use in pharmaceutical compositions includes one or more polypeptides having at least 90%, 95%, 97%, or 99% sequence identity to SEQ ID NOS-1-10, wherein the one or more polypeptides alter binding or activity of a client protein to a chaperone, co-chaperone, or chaperone-co-chaperone complex such as a specifically preselected client protein. In embodiments, the active agents for use in pharmaceutical compositions includes one or more polypeptides having at least 90%, 95%, 97% or 99% sequence identity to SEQ ID NOS-1-10, wherein the polypeptide alters binding or activity of a preselected client protein to a chaperone, co-chaperone, or chaperone-co-chaperone complex, and wherein the F(F) or 4-Fluoro-L-phenylalanine is substituted for a naturally occurring F or Phe. In embodiments, the active agents for use in pharmaceutical compositions includes one or more polypeptides comprise or consist of an amino acid sequence having 100% sequence identity to SEQ ID NOS-1-10, wherein the polypeptide alters binding or activity of a preselected or predetermined client protein to a chaperone, co-chaperone, or chaperone-co-chaperone complex, and wherein and wherein the F(F) or 4-Fluoro-L-phenylalanine is substituted for a naturally occurring F or Phe.

As disclosed herein, are pharmaceutical compositions, including any of the agents-of-interest identified in any of the methods disclosed herein and a pharmaceutical acceptable carrier described herein. In embodiments, the composition can be formulated for oral or parental administration. In embodiments, the parental administration can be intravenous, subcutaneous, intramuscular or direct injection. The compositions can be formulated for administration by any of a variety of routes of administration, and can include one or more physiologically acceptable excipients, which can vary depending on the route of administration. Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes any of the agents of interest identified using any of the methods disclosed herein dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

### Example 1

### Summary of Example 1

The chaperone heat shock protein-90 (Hsp90) controls the folding of client proteins important for tumorigenesis. The development of Hsp90 ATP-competitive inhibitors has been limited partly because it problematically results in the simultaneous blockage of all clients, ultimately causing antiapoptotic heat shock response. Herein below, the most unstable regions on the native structures of clients c-Abl, c-Src, Cdk4, B-Raf and Glucocorticoid Receptor are computationally predicted, as potential ideal interaction points with the Hsp90-system. Mimics (e.g., selective peptide inhibitors or selective peptide disruptors) were synthesized spanning these regions and confirm their interaction with partners of the Hsp90 complex (Hsp90, Cdc37 and Aha1) by Nuclear Magnetic Resonance (NMR). Designed mimics selectively disrupt the association of their respective clients with the Hsp90 machinery, leaving unrelated clients unperturbed and causing apoptosis in cancer cells. Overall, selective targeting of Hsp90 protein-protein interactions is achieved without causing indiscriminate degradation of all clients, which is useful for the development of therapeutics based on specific chaperone:client perturbation. Aspects of Example 1 are described in (Paladino et al., Targeting the folding of Hsp 90 clients by predicting their local unfolding status, Chemistry A European Journal, Vol. 26, (43) (2020) herein entirely incorporated by reference.

### Materials and Methods

Molecular Dynamic (MD) Simulations

MD simulations of all proteins described herein below were carried out using the Gromacs software package (v.4.5.5) (see for example the website at www.gromacs.org) with the Amber99 force field. Selected starting structures for protein kinases were the following: the crystal structures of the active (pdb code 2GQG) and inactive (pdb 2G1T) states of the catalytic domain of c-Abl kinase; the crystal structures of the B-Raf (pdb 4E26), cSrc (pdb 2SRC), and Cdk4 (pdb 3G33). The crystal structure used for the simulation of Glucocorticoid Receptor (GR) was 5nfp.pdb. All proteins were simulated in their apo forms. The proteins were centered in triclinic boxes allowing a 0.9 nm distance from each box edge and solvated with TIP3P water molecules. See Jorgensen et al, J. Chem Phys. 79, 926-935 (1983). Counterions were randomly added to ensure overall charge neutrality. Each system was first energy minimized using the steepest descent approach, followed by a 5 ns simulation in which the positions of the protein heavy atoms were restrained by a harmonic potential. Production trajectories were run for 100 ns at constant temperature of 300 K and a constant pressure of 1 atm. See Berendsen et al, J. Chem. Phys, 81, 3684-3690 (1984). All simulations were run in two independent replicates. A cutoff radius of 0.9 nm for non-bonded van der Waals interactions was used in all simulations. Bond lengths involving hydrogens were restrained by the LINCS algorithm. See Hess et al, J. Comp. Chem, 18, 1463-1472 (1997). Electrostatic interactions were treated using the particle mesh Ewald method. See Darden et al, J. Chem. Phys., 98, 1993. The time step was set to 2 fs and periodic boundary conditions were applied in all three dimensions.

Cluster analysis of MD trajectories was performed prior to the prediction of locally unstable structures based on the energy decomposition method. See a) Marchetti et al., J. Phys. Chem. Lett., 10, 1489-1497 (2019); b) Peri et al., ACS Chemical Biology, 8, 397-404 (2013); c) Morra et al., Proteins: Struct. Funct. and Bioinf. 72, 660-672 (2008); d) Scarabelli et al., Biophys. J., 98, 1966-1975 (2010); and e) Genoni et al., J. Phys. Chem. B., 116, 3331-3343 (2012). Clustering was carried out using 0.1 nm RMSD cut-off definition for neighbor structures using the method developed by Daura et al. Chemie Intl. Ed. 38, 236-240 (1999). The representative structures (centroid) of the first 3 clusters for each system have been analyzed for epitope prediction, and consensus on the predictions on the 3 clusters was used to select the sequences for synthesis.

### Protein Data Base (PDB) ID Numbers for Kinases of the Present Disclosure

The active (pdb:2GQG) and inactive (pdb:2G1T) states of the catalytic domain of c-Abl kinase; the crystal structures of the B-Raf (pdb:4E26), cSrc (pdb:2SRC), and Cdk4 (pdb:3G33). The crystal structure used for the simulation of Glucocorticoid Receptor (GR) was pdb:5nfp.

### Computational Prediction and Design of Chaperone/Cochaperone Targeting Regions on Client Proteins

Prediction of chaperone/cochaperone binding was carried using a matrix of local coupling energies (MLCE method) (See: a) Marchetti et al., J. Phys. Chem. Lett., 10, 1489-1497 (2019); b) Peri et al., ACS Chemical Biology, 8, 397-404 (2013); c) Morra et al., Proteins: Struct. Funct. and Bioinf. 72, 660-672 (2008); d) Scarabelli et al., Biophys. J., 98(9), 1966-1975 (2010); and e) Genoni et al., J. Phys. Chem. B., 116, 3331-3343 (2012)). based on the eigenvalue decomposition of the matrix of residue-residue energy couplings calculated for each client analyzed. Briefly, an interaction matrix Mᵢⱼ is calculated by considering the interaction energies between residue pairs, including all the non-bonded inter-residue atomic energy components (namely, van der Waals and electrostatic), in representative clusters of MD trajectory starting from the native conformation. In this calculation, diagonal elements, containing self-interactions, are neglected. The matrix Mᵢⱼ can be diagonalized and re-expressed in terms of eigenvalues and eigenvectors, in the form:

$${M}_{ij} = {\sum }_{k = 1}^{N} {λ}_{k} {w}_{i}^{k} {w}_{j}^{k} \qquad ( 1 )$$

where N is the number of amino acids in the protein, λₖ is an eigenvalue, and wⱼ^{k} is the i-th component of the associated normalized eigenvector. Eigenvalues are labelled following an increasing order, so that λₖ is the most negative. In the following we refer to the first eigenvector as the eigenvector corresponding to the eigenvalue λₖ. The total non-bonded enemy E_{nb} is defined as:

$${E}_{nb} = {\sum }_{i , j = 1}^{N} {M}_{ij} = {\sum }_{i , j = 1}^{N} {\sum }_{k = 1}^{N} {λ}_{k} {{w}_{i}}^{k} {{w}^{k}}_{j} \qquad ( 2 )$$

If the term λₖwᵢ^{k}w^{k}ⱼ for k>1 is smaller than λ₁wᵢ^{k}w^{k}ⱼ, each Mᵢⱼ can be approximated by the first contribution only:

Mᵢⱼ≈\tilde{M}ᵢⱼ=λ₁wᵢ^{j}wⱼ^{j} (3)

such that the total non bonded energy becomes:

$${E}_{nb} ≃ {E}_{nb}^{tot} = {\sum }_{i , j = 1}^{N} {\tilde{M}}_{ij} = {\sum }_{i , j = 1}^{N} {λ}_{1} {w}_{i}^{1} {w}_{j}^{1} \qquad ( 4 )$$

This simplified energy matrix captures the residue pairs contributing most to the stabilization of the overall fold, as well as the structures that are unstable and prone to support the local, large structural fluctuations that lead to unfolding. To focus on the latter, the map of pair energy-couplings corresponding to the lowest eigenvector is filtered with the contact matrix, to identify which local couplings characterized by energetic interactions of minimal intensities. Thanks to the low intensity constraints to the rest of the protein, these substructures would be characterized by dynamic properties that allow them to visit multiple conformations, a subset of which can be lead to local unfolding and be recognized by members of the Hsp90 chaperone system. The lowest 15% of all contact-filtered pairs define the (amino acid or polypeptide) residue making up the predicted chaperone/cochaperone binding sequences.

### Synthesis of Peptide-Based Mimics of Client Interaction Substructures

Peptides spanning the predicted chaperone-interaction regions of the client proteins studied here were synthesized with classical solid-phase based methods (See below).

### NMR Experiments

All the NMR experiments have been recorded at 298° K with a Bruker FT NMR Avance III 600-MHz spectrometer equipped with a 5-mm CryoProbe™ QCI¹H/¹⁹F-¹³C/¹⁵N-D quadruple resonance, a shielded z-gradient coil, and the automatic sample handling system such as a SAMPLEJET™ brand sampling system with temperature control.

¹⁹F NMR experiments are a well-recognized approach to study the interaction between small molecules or peptides and proteins. (See for example, Dalvit et al., J. Med. Chem., 62, 2218-2244 (2018)). ¹⁹F NMR shows one of the largest relative sensitivity to protein binding events. This is due to the large dynamic range defined as the difference of the NMR measured response in the free and protein-bound states. ¹⁹F R₂ filter NMR experiments are among the most sensitive techniques for weak binding detection. See Dalvit et al., Journal of American Chemical Society, 125, 7696-7703 (2003). The transverse relaxation rate R₂ is a very sensitive parameter for these studies, due to the large Chemical Shift Anisotropy (CSA) of ¹⁹F nucleus and to the large exchange contribution See Dalvit et al., Journal of American Chemical Society, 125, 7696-7703 (2003); the compounds/peptides that interact with the receptor will show a broadening and intensity reduction in their ¹⁹F NMR signal in presence of the protein

The 5 mM stock solution peptides were prepared in 100% in DMSOd6. Solubility and purity of the peptides in PBS buffer pH 7.4, 10% D₂O (for the lock signal) were checked by ¹⁹F and ¹H NMR spectroscopy. 1D ¹⁹F NMR experiments were recorded with proton decoupling with the Waltz-16 scheme during the acquisition period with an acquisition time of 0.95 s, a relaxation delay of 30 s. whereas 1D version of the NOESY (nuclear Overhauser effect spectroscopy) pulse sequence with H₂O signal presaturation, a mixing time of 10 ms and a relaxation delay of 30 s was used for ¹H NMR experiments.

For the binding studies R₂ filter experiments were recorded with the Carr-Purcell-Meibom-Gill scheme with a time interval of 23.5 ms between the 180° pulses with a loop of 2, an acquisition time of 0.95 s a D1 of 5 s and a number of scans of 512. All the ¹⁹F chemical shifts are referenced to the CFCl₃ signal in water.

### Mammalian Cell Culture

Human embryonic kidney (HEK293) and 786-O cells were acquired from the American Type Culture Collection (ATCC). HEK293 cells were grown in Dulbecco's Modified Eagle Medium (DMEM, Millipore-Sigma) and 786-O cells were grown in Roswell Park Memorial Institute (RPMI-1640, Millipore-Sigma) medium supplemented with 10% fetal bovine serum (FBS, Millipore-Sigma) in a CellQ incubator (Panasonic Healthcare) at 37° C. in 5% CO2.

### Peptide Treatment

Cultured cells were seeded 24 h prior to treatment. Peptides were added to cells at 50% confluency at the indicated concentrations and incubated for 24 h, followed by protein extraction as described below.

### Protein Extraction, Immunoprecipitation and Immunoblotting

Protein extraction from mammalian cells was carried out using methods previously described. See Woodford et al., Cell Reports, 14, 872-884 (2016). For immunoprecipitation, protein lysates were incubated with Hsp90 antibody for 2 h followed by incubation with protein G agarose (Qiagen) for 2 h at 4° C. Immunopellets were washed 4 times with fresh lysis buffer (20 mM Tris (pH7.4), 100 mM NaCl, 1 mM MgCl2, 0.1% NP40, protease inhibitor cocktail (Roche), and PhosSTOP (Roche)) and eluted with 5× Laemmli buffer. Precipitated proteins were separated by SDS-PAGE and transferred to nitrocellulose membranes. Co-immunoprecipitated proteins were detected by immunoblotting with the indicated antibodies, diluted in 5% non-fat dry milk reconstituted in TBST.

### Biotinylated Peptide Pulldown

Total cell lysates prepared as described above were incubated with the indicated amounts of biotinylated peptide at 4° C. for 1 h. Streptavidin agarose beads (ThermoScientific) were added and incubated 1 additional hour with gentle rotation. Bound Hsp90 was detected by immunoblotting as described herein.

### Fluorescence Imaging

FAM-labeled peptides were incubated with cultured cells for 24 h. Brightfield and fluorescent images were captured using the ZOE Fluorescent Cell Imager (Bio-Rad).

### Peptide Synthesis and Characterization

### Materials

HMPB resin, N-α-Fmoc-L-amino acids and building blocks used during chain assembly were purchased from Iris Biotech GmbH (Marktredwitz, Germany). Ethyl cyanoglyoxylate-2-oxime (Oxyma) was purchased from Novabiochem (Darmstadt, Germany), N,N′-dimethylformamide (DMF) and trifluoroacetic acid (TFA) were from Carlo Erba (Rodano, Italy). N,N′-diisopropylcarbodiimide (DIC), dichloromethane (DCM) and all other organic reagents and solvents, unless stated otherwise, were purchased in high purity from Sigma-Aldrich (Steinheim, Germany). All solvents for solid-phase peptide synthesis (SPPS) were used without further purification. HPLC grade acetonitrile (ACN) and ultrapure 18.2Ω water (Millipore-MilliQ) were used for the preparation of all buffers for liquid chromatography. The chromatographic columns were from Phenomenex (Torrance Calif., USA). HPLC eluent A: 97.5% H₂O, 2.5% ACN, 0.7% TFA; HPLC eluent B: 30% H₂O, 70% ACN, 0.7% TFA

### Peptide Synthesis: General Procedures

### Resin Loading

Resin (0.5 mmol/g loading) was swollen in CH₂Cl₂ for 30 min then washed with DMF (3×3 mL). A solution of entering Fmoc-amino acid, DIC and Oxyme (5:5, 5 eq over resin loading) and 5% of DMAP in DMF (3 mL) was added and the resin shaken at rt for 4 h. The resin was washed with DMF (2×3 mL) and capping was performed by treatment with acetic anhydride/DIEA in DCM (1×30 min). The resin was then washed with DMF (2×3 mL), CH₂Cl₂ (2×3 mL), and DMF (2×3 mL). The resin was subsequently submitted to fully automated iterative peptide assembly (Fmoc-SPPS).

### Peptide Assembly Via Iterative Fully Automated Microwave Assisted SPPS

Peptides were assembled by stepwise microwave-assisted Fmoc-SPPS on a Biotage ALSTRA Initiator+peptide synthesizer, operating in a 0.1 mmol scale. Activation of entering Fmoc-protected amino acids (0.3M solution in DMF) was performed using 0.5M Oxyma in DMF/0.5M DIC in DMF (1:1:1 molar ratio), with a 5 equivalent excess over the initial resin loading. Coupling steps were performed for 7 minutes at 75° C. Fmoc-deprotection steps were performed by treatment with a 20% piperidine solution in DMF at room temperature (1×10 min). Following each coupling or deprotection step, peptidyl-resin was washed with DMF (4×3.5 mL). Upon complete chain assembly, resin was washed with DCM (5×3.5 mL) and gently dried under a nitrogen flow.

Cleavage from the Resin

Resin-bound peptide was treated with an ice-cold TFA, TIS, water, thioanisole mixture (90:5:2.5:2.5 v/v/v/v, 4 mL). After gently shaking the resin for 2 hours at room temperature, the resin was filtered and washed with neat TFA (2×4 mL). The combined cleavage solutions were worked-up as indicated below.

### Work-Up and Purification

Cleavage mixture was concentrated under nitrogen stream and then added dropwise to ice-cold diethyl ether (40 mL) to precipitate the crude peptide. The crude peptide was collected by centrifugation and washed with further cold diethyl ether to remove scavengers. Residual diethyl ether was removed by a gentle nitrogen flow and the crude peptide was purified by RP-HPLC and lyophilized.

### Synthesis of Fluorescein-Labelled Peptides

Cysteine-bearing peptides were conjugated to bifunctional MAL-FAM (Lumiprobe GmbH, Germany) as follows: peptide (1 eq.) was dissolved in phosphate buffer (Na₂HPO₄ 0.4M, pH 7.8). The resulting solution was ice-cooled and mixed with MAL-FAM solution (1.2 eq., 50:50 acetonitrile/water mixture). The reaction mixture was left to react for under gentle shaking until full reagents conversion (RP-HPLC monitoring). Upon reaction completion, conjugation products were isolated by preparative RP-HPLC and lyophilized.

### RP-HPLC Analysis and Purification

Analytical RP-HPLC was performed on a Shimadzu Prominence HPLC (Shimadzu) using a Shimadzu Shimpack GWS C18 column (5 micron, 4.6 mm i.d.×150 mm). Analytes were eluted using a binary gradient of mobile phase A (100% water, 0.1% trifluoroacetic acid) and mobile phase B (30% water, 70% acetonitrile, 0.1% trifluoroacetic) using the following chromatographic method: 10% B to 100% B in 14 min; flow rate, 1 ml/min.

Preparative RP-HPLC was performed on a Shimadzu HPLC system using a Shimadzu C18 column (10 micron, 21.2 mm i.d.×250 mm) using the following chromatographic method: 0% B to 100% B in 45 min; flow rate, 14 ml/min. Pure RP-HPLC fractions (>95%) were combined and lyophilized.

### Electro-Spray Ionization Mass Spectrometry (ESI-MS)

Electro-spray ionization mass spectrometry (ESI-MS) was performed using a Bruker Esquire 3000+ instrument equipped with an electro-spray ionization source and a quadrupole ion trap detector (QITD).

TABLE 1
Peptide list

| Code | Sequence |
|---|---|
| A01 | LGGGQF(F)GEVYGGVAVKTLGGGEFLDEAAVMK |
| | (SEQ ID NO: 1) |
| A02 | F(F)GGSPYPGIDLSQVYELLEK |
| | (SEQ ID NO: 2) |
| B-Raf_01 | GYSTKPQLAGGGNVTAPTPQGF(F)QHSGS |
| | (SEQ ID NO: 3) |
| B-Raf_01 | FGTVYKGKWGGGGF(F)STKPQLAGGGNVTAPTPQ |
| | (SEQ ID NO: 4) |
| Cdk4_01 | CATSRTDREGPNGGGGGGGLPISTGGF(F)QMALTPVV |
| | (SEQ ID NO: 5) |
| Cdk4_02 | PVAEIGVGAYGGGRVPGGF(F)QMALTPVV |
| | (SEQ ID NO: 6) |
| cSrc_01 | LGQGCF(F)GGKPGTMSPGGEEPGGRESLGWNGTT |
| | (SEQ ID NO: 7) |
| cSrc_02 | GEMGKGGKGRVPYPGMVNREVLDQVERGF(F)RM |
| | (SEQ ID NO: 8) |
| GR-01 | TLPCGGTWRIMTGIE F(F)PEMLA |
| | (SEQ ID NO: 9) |
| GR-02 | YAGYDSSVPDSTWRIMTTLNMGGF(F)PEMLA |
| | (SEQ ID NO: 10) |

F(F) refers to one phenylalanine characterized as 4-Fluoro-L-phenylalanine (e.g. including a protecting group). In embodiments, any peptide protecting group can be included. In some embodiments, the F(F) may be substituted for a naturally occurring F or Phe. In embodiments, SEQ ID NOS: 1-10 do not include F(F), but only include F in place of F(F).

TABLE S2
Peptide characterization

| Code | ESI-MS (m/z) found | | ESI-MS (m/z) calculated | | | Rt | |
|---|---|---|---|---|---|---|---|
| A01 | 1031.3 | (M³⁺), | 1031.2 | (M³⁺), | 1546.3 | 11.2 | min |
| | 1546.9 (M²⁺) | | (M²⁺) | | | | |
| A02 | 1116.2 (M²⁺) | | 1116.7 | (M²⁺) | | 12.05 | min |
| B-Raf_01 | 1303.7 (M²⁺) | | 1303.5 | (M²⁺) | | 8.3 | min |
| B-Raf_02 | 1592.9 | (M²⁺), | 1593.7 | (M²⁺), | 1062.8 | 9.1 | min |
| | 1062.3 (M³⁺) | | (M³⁺) | | | | |
| Cdk4_01 | 1677.9 (M²⁺) | | 1677.8 | (M²⁺) | | 9.8 | min |
| Cdk4_02 | 2634.5 (M⁺), 1318.0 | | 2634.2 | (M⁺), | 1317.7 | 11.7 | min |
| | (M²⁺) | | (M²⁺) | | | | |
| cSrc_01 | 1579.1 | (M²⁺), | 1579.5 | (M²⁺), | 1053 | 8.9 | min |
| | 1052.8 (M³⁺) | | (M³⁺) | | | | |
| cSrc_02 | 1735.3 | (M²⁺), | 1735.1 | (M²⁺), | 1157.1 | 10.1 | min |
| | 1157.3 (M³⁺) | | (M³⁺), | | | | |
| GR01 | 1172.6 (M²⁺) | | 1172.3 | (M²⁺) | | 12.56 | min |
| GR02 | 1116.5 (M²⁺) | | 1116.3 | (M²⁺) | | 12.52 | min |
| B-Raf_01_FAM | 1070.5 | (M³⁺), | 1070.3 | (M³⁺), | 1604.8 | 9.9 | min |
| | 1605.2 (M²⁺) | | (M²⁺) | | | | |
| B-Raf_02_FAM | 1263.1 | (M³⁺), | 1894.8.5 | (M²⁺), | 1263.5 | 10.4 | min |
| | 1895.0 (M²⁺) | | (M³⁺) | | | | |
| Cdk4_01_FAM | 1925.4 | (M²⁺), | 1925.5 | (M²⁺), | 1284.5 | 11.2 | min |
| | 1284.7 (M³⁺), | | (M³⁺) | | | | |
| Cdk4_02_FAM | 1618.5 (M²⁺) | | 1618.6 | (M²⁺) | | 12.1 | min |
| cSrc_01_FAM | 1419.3 | (M³⁺), | 1419.6 | (M³⁺), | 1065 | 11.3 | min |
| | 1064.8 (M⁴⁺) | | (M⁴⁺) | | | | |
| cSrc_02_FAM | 1373.7 | (M³⁺), | 1373.6 | (M³⁺), | 1030.5 | 11.9 | min |
| | 1030.9 (M⁴⁺) | | (M⁴⁺) | | | | |

### Example I Introduction

The Hsp90 family of molecular chaperones plays key roles in cell proteostasis by balancing the folding, activation, and turnover of a diverse set of client proteins, many of which are fundamental for cancer development. Hsp90 functions depend on ATP hydrolysis and interactions with clients and co-chaperones. Hsp90 inhibition by ATP competitive inhibitors, however, leads to the indiscriminate depletion of all Hsp90 clients, thereby causing the upregulation of the heat shock response which ultimately protects cancer cells from apoptosis and causes toxicity.

As mentioned above, the formation of client:Hsp90 complexes is a critical step in the regulation of specific client activities. Specificity in the selection of clients that lack sequence and structural homology is acquired through recruiter cochaperones that provide the essential recognition/discrimination elements. In this framework, Hsp90, its cochaperones and the clients engage in multicomponent assemblies, stabilized by dynamic protein-protein interactions (PPIs). Co-chaperones such as Cdc37 control the entry of kinases and other clients into the chaperone cycle, while other co-chaperones, such as Aha1, provide additional layers of regulation by modulating the rates of ATP hydrolysis. No specific structural elements or surface characteristics have been proposed as Hsp90-binding determinants. Critical to Hsp90 mechanisms is that the interactions involved are conformationally heterogeneous, short-lived and relatively weak, with different clients interacting in distinct ways. (See e.g, Pricer et al., Accounts of chemical research 2017, 50, 584-589, Cesa et al., Front Bioeng Biotechnol 2015, 3, 119 and Thompson et al., ACS Chem. Biol. 2012, 7, 1311-1320. These observations unveil a new opportunity for the design of client-selective chemical tools based on the idea that perturbing the (weak) interactions with (any of) the members of the chaperone assembly can impair a client's folding. The lack of consensus binding motifs suggests the possibility to target unique interaction surfaces in order to specifically disrupt key client:chaperone or client:cochaperone interactions.

Here, interaction interfaces of different clients were predicted, based only on the structure of the isolated client, as the rational basis for the design of selective peptide inhibitors of protein-protein interactions in chaperone complexes. The design is based on a novel computational method developed for the prediction of locally unstable substructures in proteins. Unstable substructures represent potential ideal points of interaction with the Hsp90 machinery. This knowledge is translated into the development of peptides spanning the predicted interaction sites with the aim to engage different constituents of the Hsp90 complex (Hsp90, Cdc37, Aha1). The ability of the designed molecules to bind to their chaperone complex members was confirmed by NMR. The peptides were cell permeable and selectively interfered with the association of their respective clients with the Hsp90 chaperone machinery, ultimately causing apoptosis in cancer cells. To the best of our knowledge, our work represents the first example in which ab initio, physics-based characterization of protein stability is leveraged for the selective chemical targeting of chaperone:client interactions in multicomponent complexes. This is achieved without significant indiscriminate inhibition or degradation of all clients, setting the stage for the definition of the pharmacophoric requirements for the development of PPI targeting molecules with therapeutic potential.

### Computational Design and NMR-Characterization.

The Hsp90 system acts on clients late in their folding pathway and associates with substrates in which large parts of the domains are already folded in their native conformation. As shown herein the chaperone system targets client substructures with minimal structural stability in the native state. From the physico-chemical point of view, locally-unstable substructures are characterized by distinct energetic properties as they are not involved in major intramolecular stabilizing interactions with other regions of the protein. Minimal intramolecular coupling, in turn, favors local instability and structural variations, distinctive properties of local unfolding.

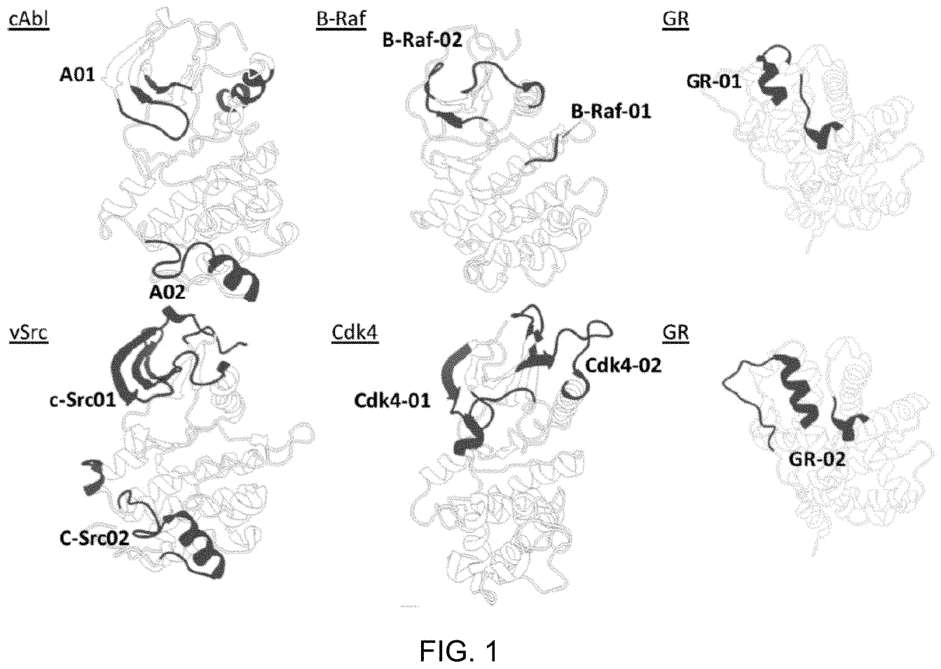

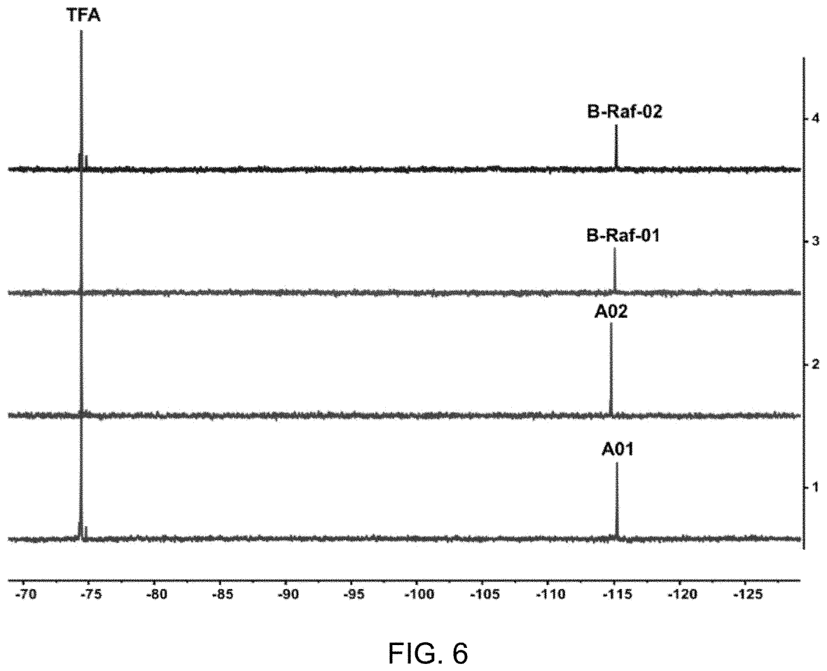

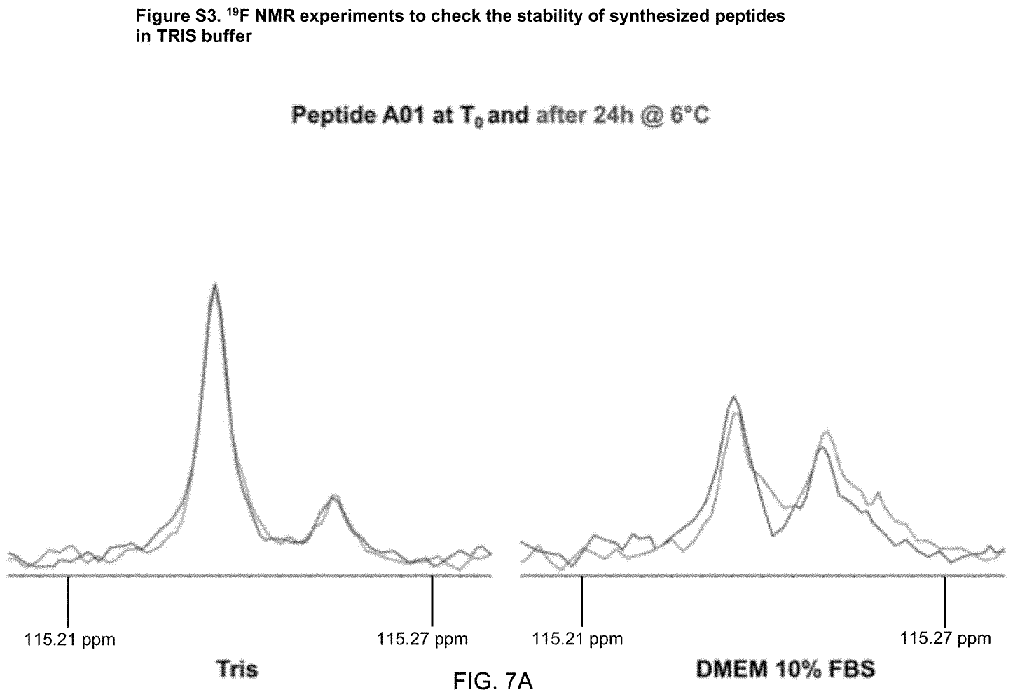

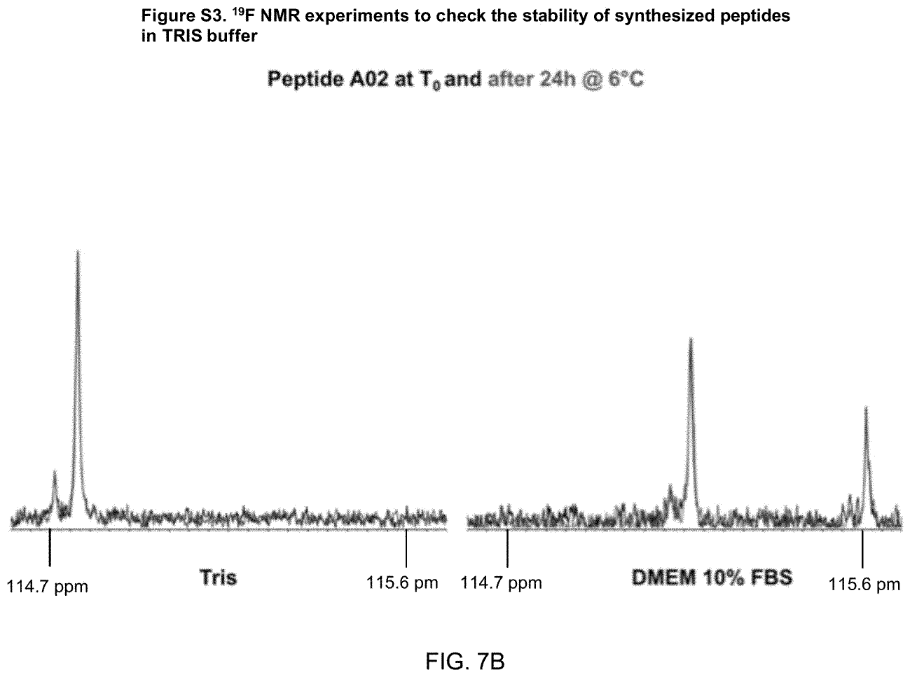

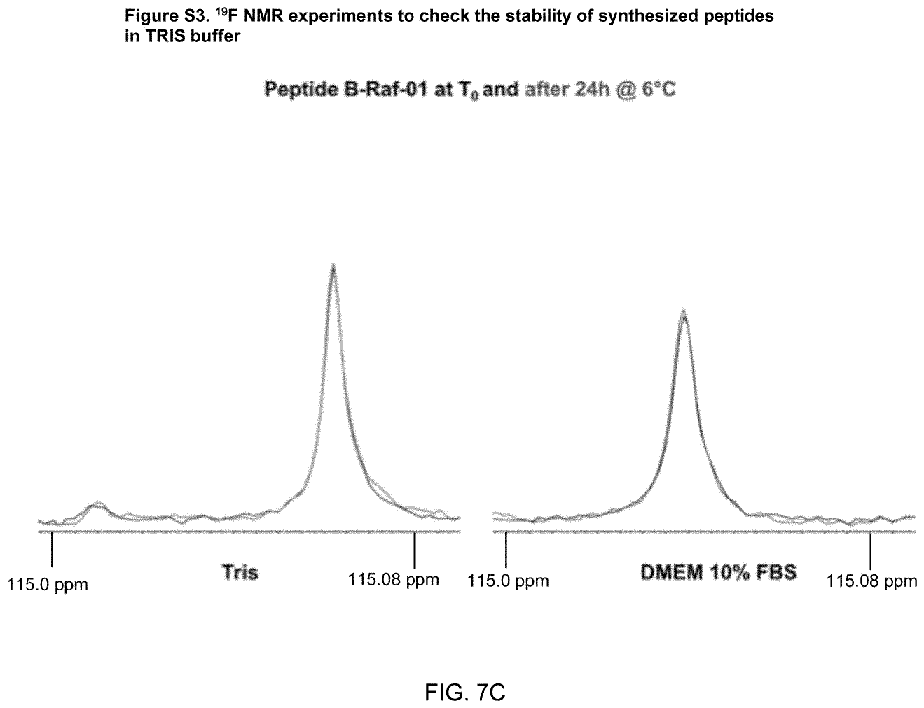

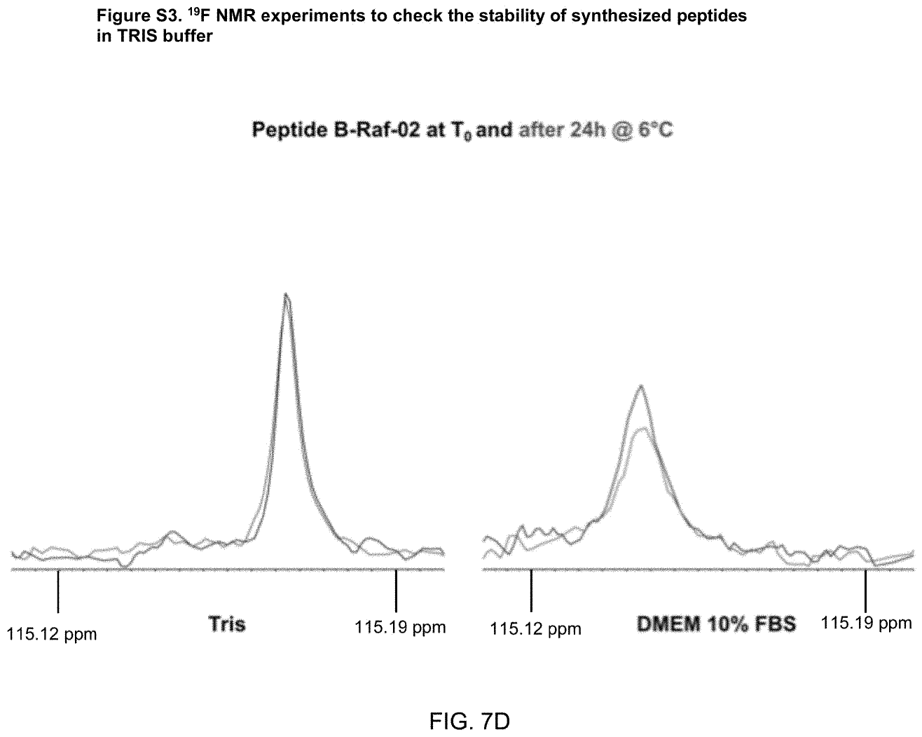

To predict the location of minimally coupled, locally unstable substructures, the MLCE method (See e.g. Marchetti et al., J. Phys. Chem. Lett. 2019, 10, 1489-1497; Peri et al., ACS Chemical Biology 2013, 8, 397-404; Morra et al., Proteins: Struct. Funct. and Bioinf. 2008, 72, 660-672 (all of which are entirely incorporated by reference)) was used. The approach was tested on the Hsp90 client Abelson leukemia (c-Abl) kinase protein. Two consensus sequences, labeled respectively A01 (SEQ ID NO: 1) and A02 (SEQ ID NO: 2) (See FIG. 1 and Table 1), were designed as potential interaction epitopes by applying MLCE to MD simulations of c-Abl. A01 (SEQ ID NO: 1) represents a conformational epitope localized at the N-lobe at the border with the C-lobe, spanning parts of the Gly-rich loop, β1, β2 and β3 strands and the αC helix (FIG. 1). To achieve optimal spanning of predicted interaction regions, the different constituent subparts were linked by the addition of a number of glycine residues approximating the average distance between the respective terminals calculated from MD simulations (see Table 1). The second epitope, A02, is linear, located at the C-lobe, and spans the aG-helix preceded by the flexible aF loop. An ¹⁹F-modified phenylalanine was site-specifically introduced into A01 and A02 to allow the characterization of the binding to members of the Hsp90 chaperone complexes (Hsp90, Cdc37, Aha1) by Fluorine Nuclear Magnetic Resonance (¹⁹F NMR) (See e.g., Dalvit, M. Flocco, M. Veronesi, B. J. Stockman, Comb. Chem. & HTS 2002, 5, 605-611 and Dalvit, P. E. Fagerness, D. T. A. Hadden, R. W. Sarver, B. J. Stockman, Journal of the American Chemical Society 2003, 125, 7696-7703) (see also Table 1). Human Serum Albumin (HSA) was used as a control for non-specific binding. The purity and stability of peptides in plasma was also checked by NMR (See FIG. 6 and FIG. 7).

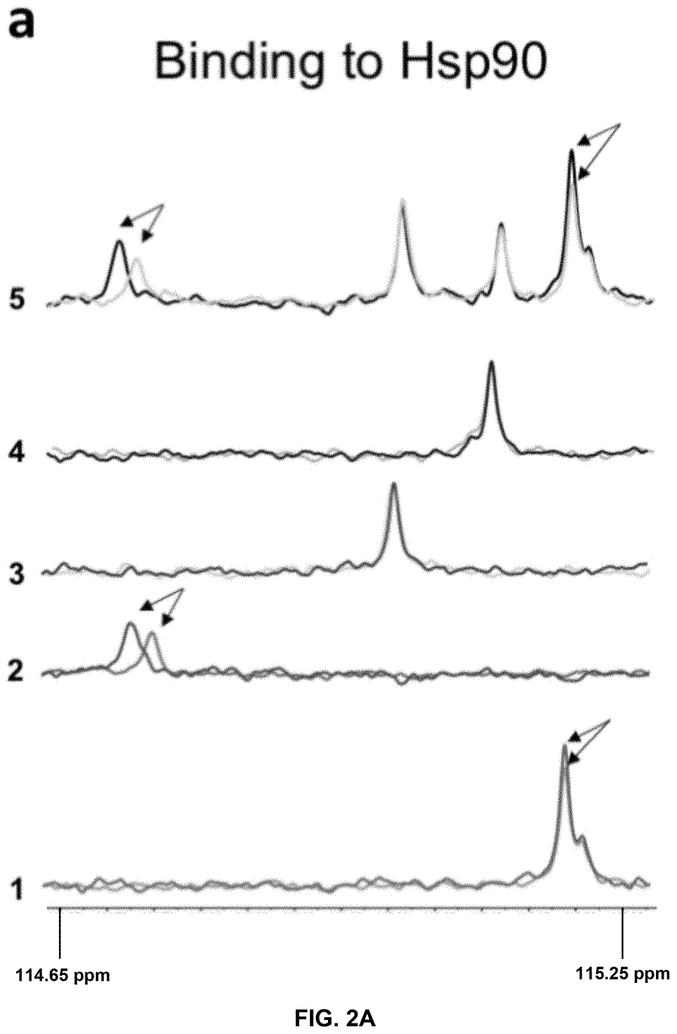

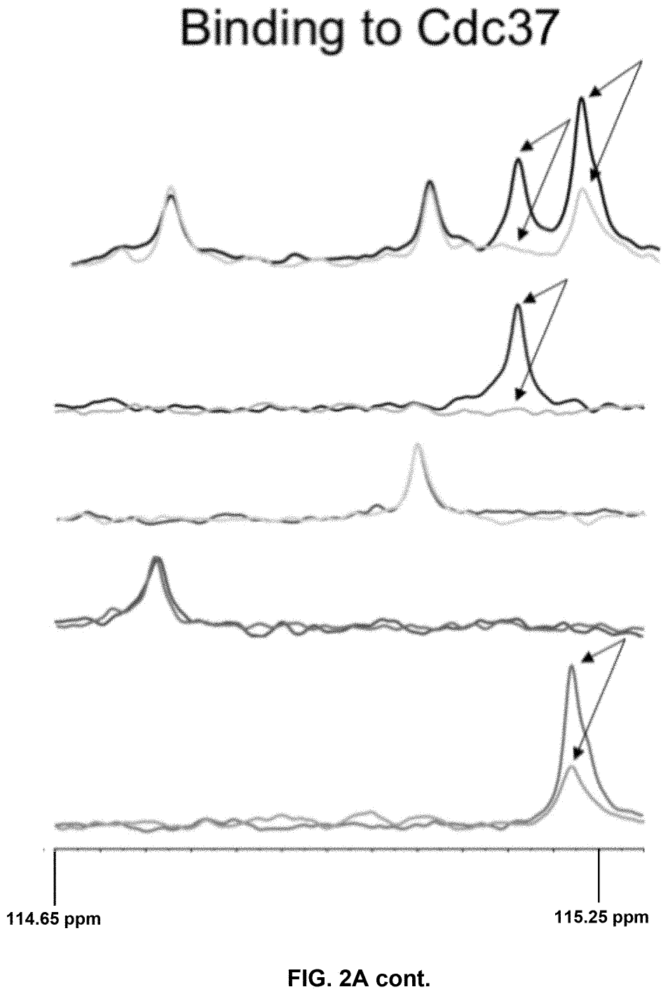

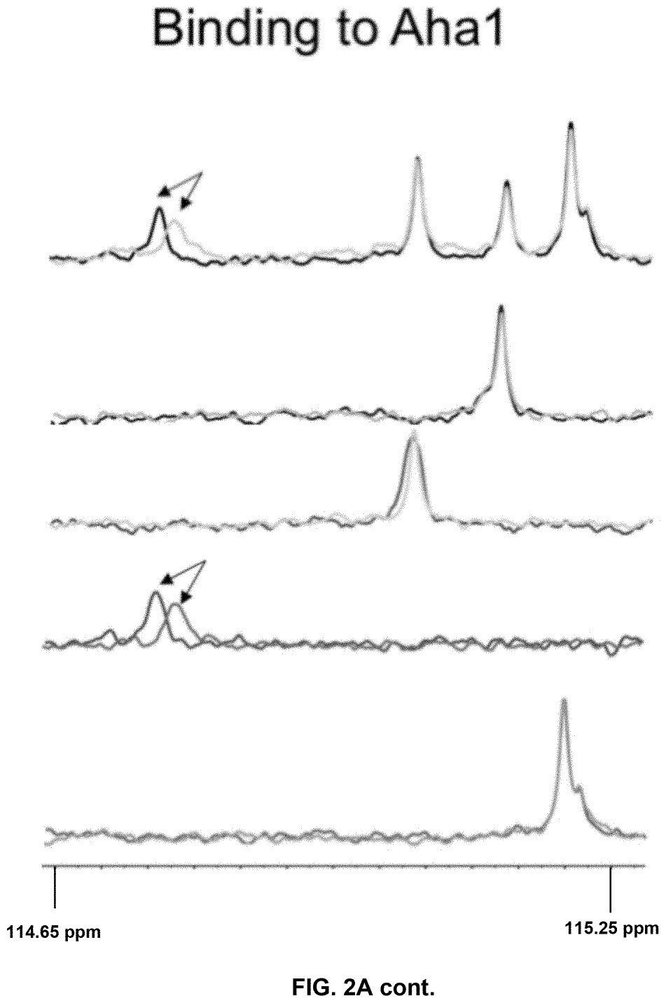

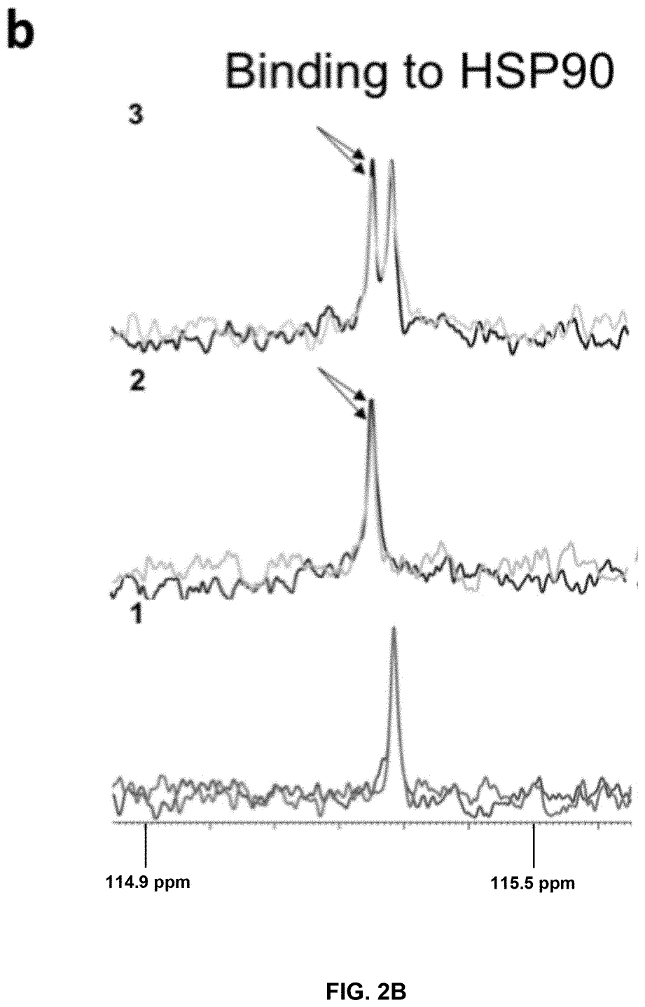

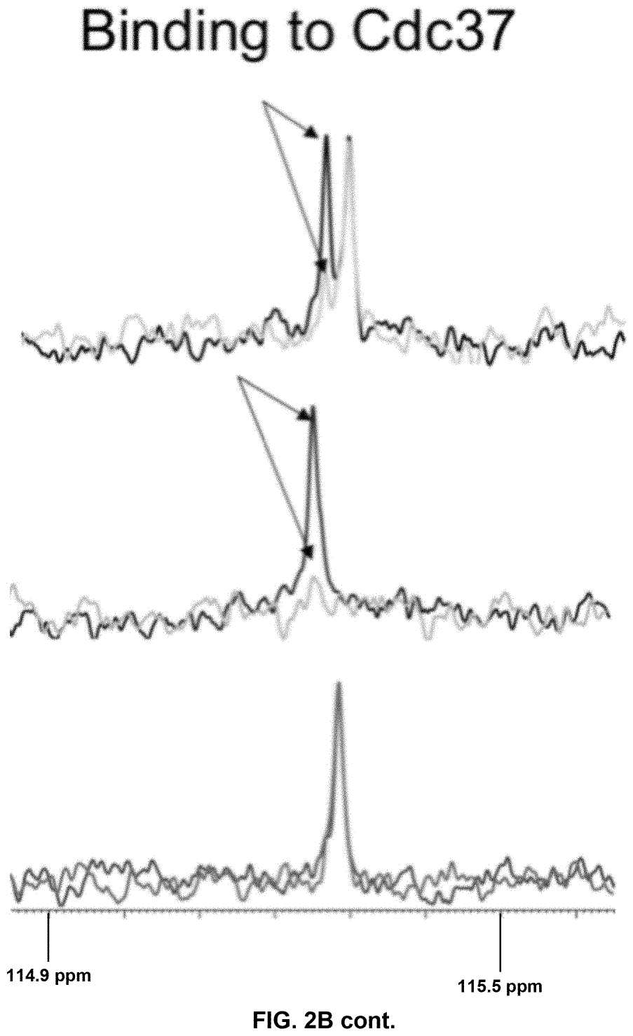

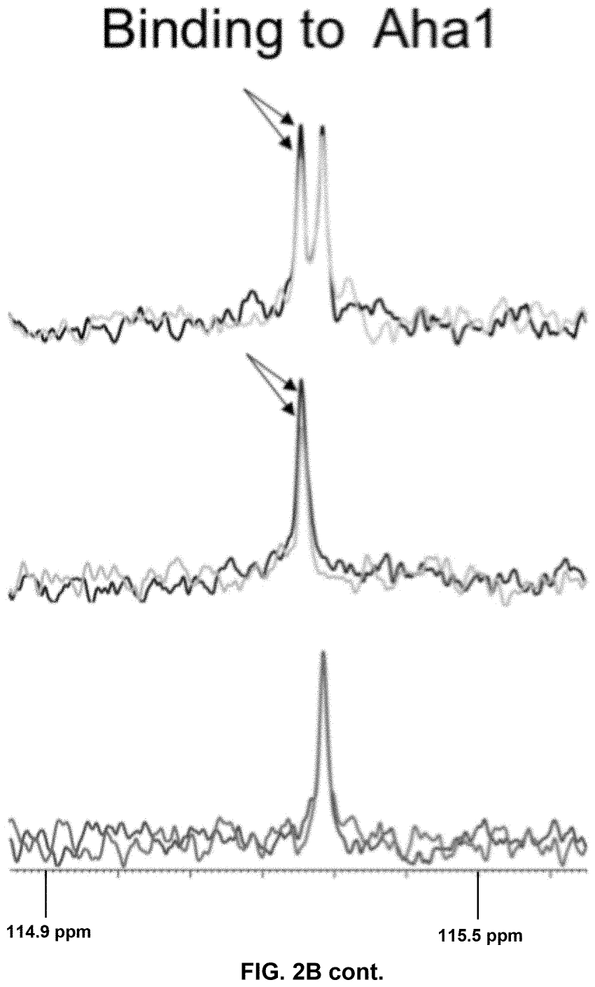

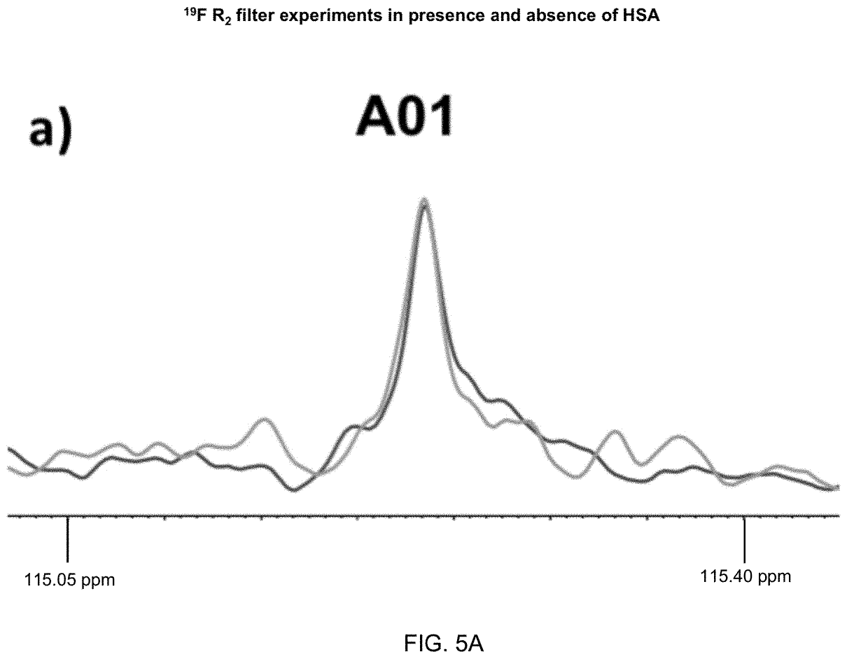

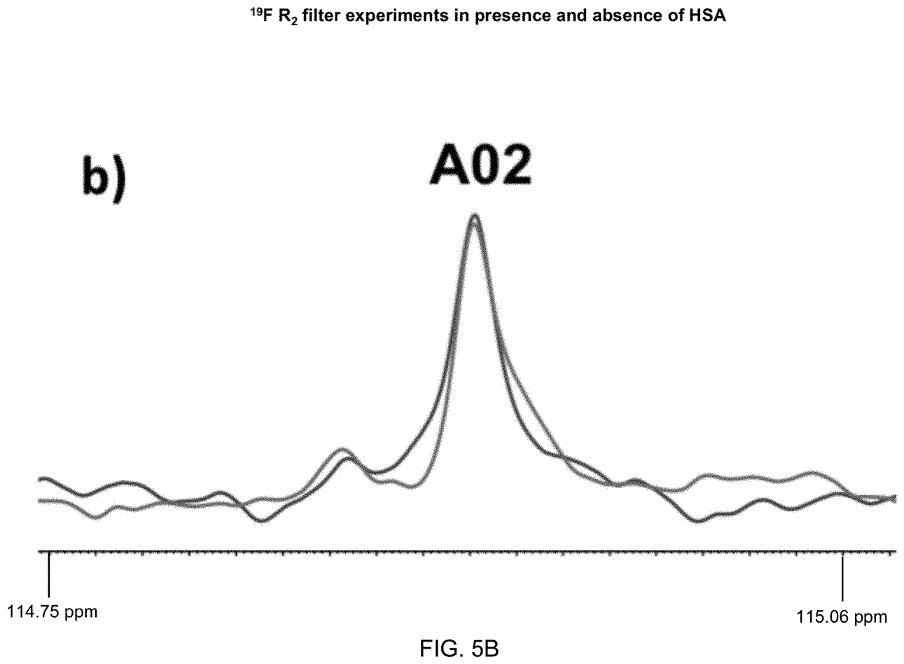

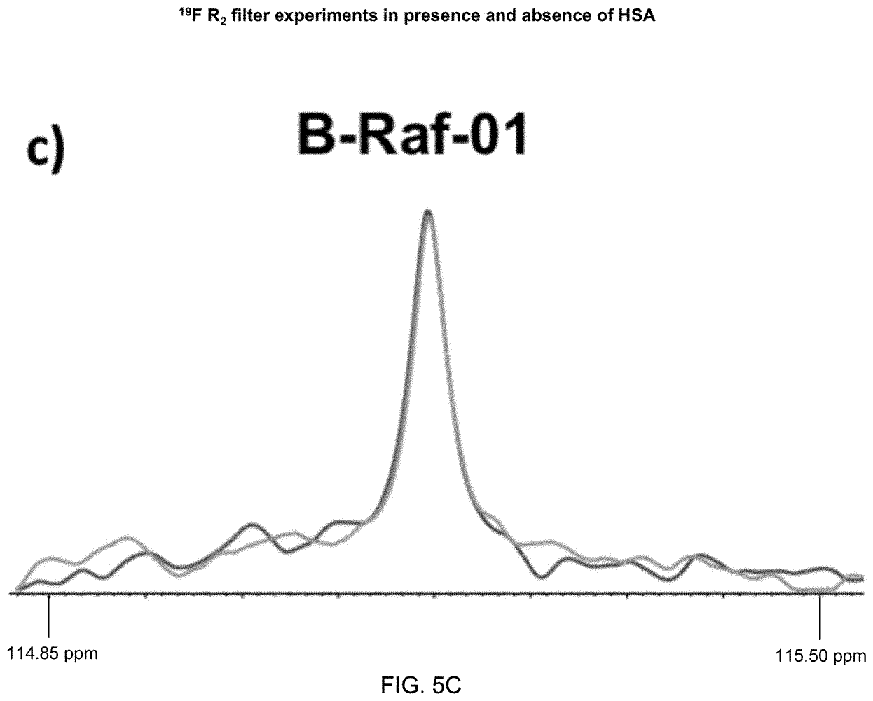

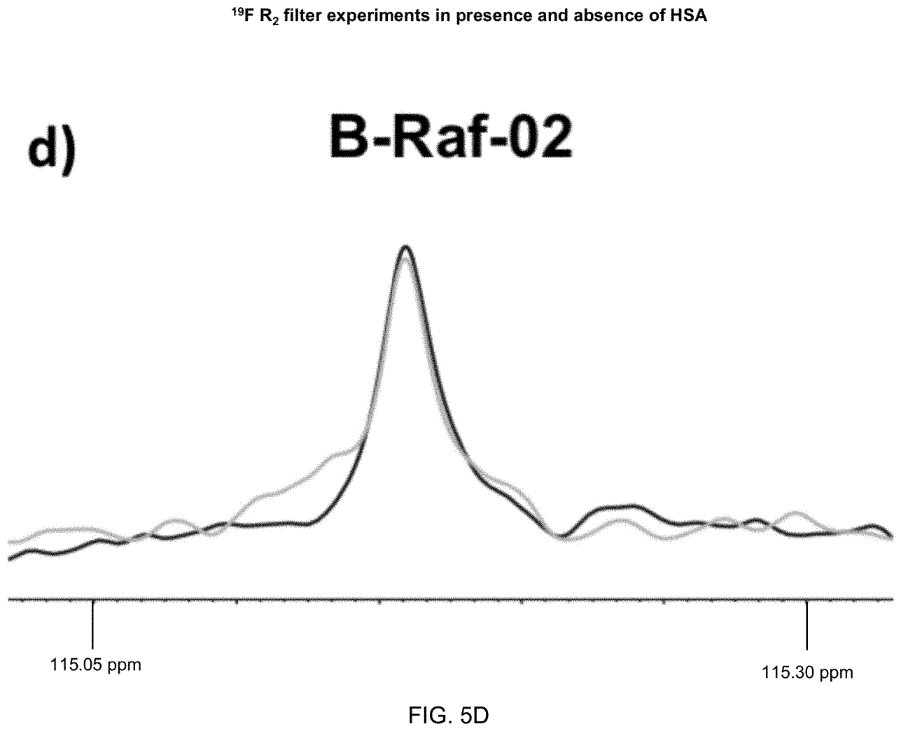

First, the peptides were tested individually at 10 mM in the presence and in absence of full length human Hsp90 by ¹⁹F R₂ filter experiments. The ¹⁹F NMR signals of A01 and A02 decrease in the presence of Hsp90, indicating peptide-protein binding (FIG. 2A, traces 1 and 2). Interestingly A01 and A02 continue to bind to Hsp90 even when they are mixed, suggesting that they do not compete with each other and interact with different sites. Next, interaction was examined by ¹⁹F-NMR of 10 mM of each cAbl peptide with Aha1 and Cdc37. A01 showed the ability to bind Cdc37 but not Aha1. 40 mM A01 and 40 mM A02 were bound with 10 mM HSA: importantly, neither A01 nor A02 showed binding interaction with HSA (See FIGS. 5A-5D).

To investigate the generality of the strategy, mimics were designed of the chaperone-binding regions of oncogenic kinase B-Raf and extended MLCE-predictions to glucocorticoid receptor (GR), a protein with structure, substrate and functions that are completely unrelated to kinases. In B-Raf, two conformational epitopes were predicted, labeled B-Raf-01 (SEQ ID NO: 3) and B-Raf-02 (SEQ ID NO: 4) (FIG. 1, Table 1), located in the N-lobe of the kinase at the border with the C-lobe. Interestingly, while the ¹⁹F NMR signals of peptides B-Raf-01 and B-Raf-02 (FIG. 2A, traces 3 and 4) did not show any difference in the presence of Hsp90, indicating no or very weak interaction, B-Raf-02 bound to Cdc37 (FIG. 2A, trace 4). Importantly, no interactions were detected-with HSA (see FIGS. 5A-5D).

In GR, two substructures were predicted (FIG. 1, Table1). Interestingly, they correspond to the region that Kirschke et al. Cell, 2014, 157, 1685-1697.previously observed to engage the Hsp90 complex. ¹⁹F-NMR confirmed binding of synthetic mimics of GR unfolding regions to the members of the chaperone complex. GR-02, in particular, bound to all of the tested proteins, with the highest effect on Cdc37 (FIG. 2, trace 2).

Because NMR data supported the viability of the prediction and design approach, the analysis of the present disclosure was extended to oncogenic kinases Cdk4 and c-Src for further testing in cancer cells. In Cdk4, the unfolding regions localize mainly in the N-lobe, corresponding to the region undergoing unfolding in the Agard cryoEM structure See K. A. Verba, R. Y. Wang, A. Arakawa, Y. Liu, M. Shirouzu, S. Yokoyama, D. A. Agard, Science 2016, 352, 1542-1547. In c-Src, an additional epitope traces the G4 helix in the C-lobe (see FIG. 1, Table 1).

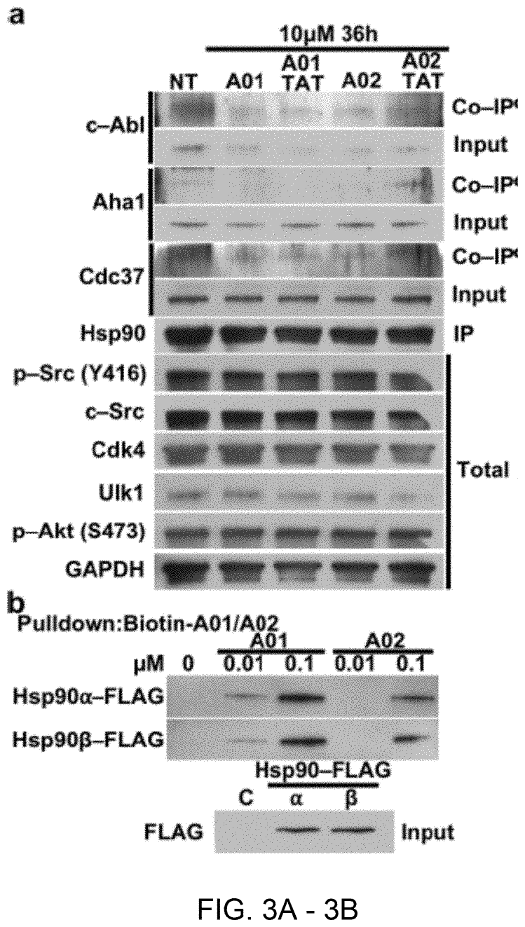

Effect of c-Abl peptides on Hsp90 chaperone complex in human cells. To demonstrate the impact of the peptides based on c-Abl-Hsp90 interactions, HEK293 cells were treated with A01 and A02. A decrease of c-Abl protein levels and dissociation of c-Abl from Hsp90 was demonstrated, consistent with interference of Hsp90 chaperoning by A01 and/or A02 (FIG. 3A). Importantly, neither the related kinase c-Src nor the client kinases Cdk4 or Akt showed a defect in stability or activity, indicating the specificity of the A01 and A02 designed peptide epitopes for c-Abl (FIG. 3A). c-Abl phosphorylates Aha1-Y223 and promotes its binding to Hsp90 (See Dunn et al., Cell Reports 2015, 12, 1006-1018). A01 and A02 treatment decreased Aha1-Hsp90 complex formation, demonstrating that the peptide-mediated dissociation of c-Abl and Hsp90 elicits a functional consequence (FIG. 3a). Whether A01 and A02 preferred a particular isoform of Hsp90, the constitutively expressed Hsp90β or the stress-inducible Hsp90α was tested. Streptavidin-pulldown of the biotinylated peptides shows A01 binds with higher affinity to both isoforms of Hsp90 but neither peptide demonstrates isoform specificity (FIG. 3b). Taken together it was demonstrated that c-Abl-mimic peptides can disrupt the specific interaction of this client kinase with Hsp90 in cells.

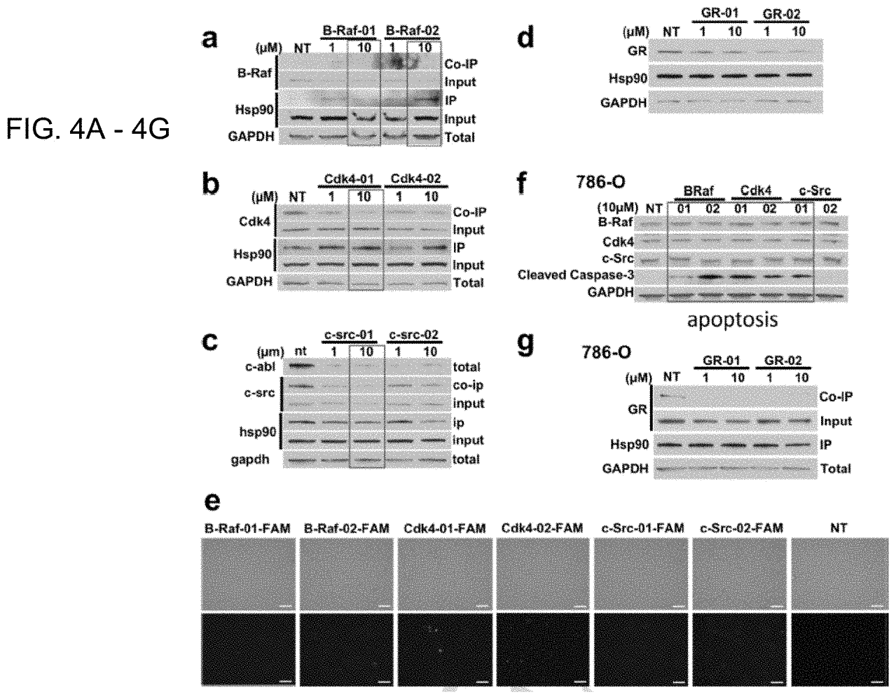

Physiological impact of Hsp90 client-based peptides. The data with the c-Abl-based inhibitors prompted an examination of the impact of additional peptides spanning the predicted interaction regions of other clients (B-Raf, Cdk4, c-Src and GR) on their respective client:chaperone interactions. Treating HEK293 cells with increasing concentrations of these peptides led to dissociation of the clients from Hsp90 (FIG. 4a-d).

The peptides labeled with fluorescein can readily enter HEK293 cells (FIG. 4E). The effect of these peptides in cancer cells was examined. The clear cell renal cell carcinoma cell line 786-O were treated with B-Raf, Cdk4, and c-Src-based inhibitors. All the mimicking peptides tested, except c-Src02, induced apoptosis, as revealed by the elevation of the apoptotic marker cleaved caspase-3 (FIG. 4F). Finally, the 786-O cells were treated with GR-based inhibitors (GR-01 and GR-02) and showed GR dissociation from Hsp90 (FIG. 4g). Taken together, these data indicate that the designed peptides have the ability to enter cells and dissociate client proteins from Hsp90. They also demonstrate biological activity as they induce apoptosis in a cancer cell line.

### DISCUSSION AND CONCLUSIONS

An integrated approach has been developed combining ab initio design, synthesis and cellular testing to advance the mechanistic investigation of protein stability. The strategy designed molecules that selectively interfere (e.g., selective peptide inhibitors) with Hsp90-mediated protein folding. The data shows that predicted locally unstable regions from the native structures of clients can define the preferential points of interaction with members of the Hsp90 system and can be mimicked by synthetic peptide agents spanning such regions. These rationally designed chemical tools induce selective dissociation and/or degradation of cognate clients in cells.

Here, the present disclosure provides for the concept of selective targeted protein depletion based on the interference with the protein-protein interactions (PPI) that underpin Hsp90-mediated folding processes. Binding of the synthetic peptide agents to Hsp90 and/or its cochaperones Cdc37 and Aha1 interferes with the formation of the complexes that control protein conformational maturation. (See also Zierer, et al., Nat Struct Mol Biol 2016, 23, 1020-1028, and Li et al., Nature structural & molecular biology 2011, 18, 61-66. It is worth noting that the affinities between Hsp90, cochaperones and clients are weak and only when all the components are correctly assembled do the complexes become functional. The client-mimicking PPI inhibitors can specifically target Hsp90 and/or Cdc37/Aha1, disrupt the binding of the clients to the chaperone complex and significantly reduce their cellular levels, likely due to their degradation.

At the atomic level, the EM structure from the Agard lab shows the N- and C-lobes of Cdk4 in complex with Hsp90-Cdc37 are completely separated, with the hinge region including the αC helix largely unfolded^{[13b]}. Importantly, the location of the hinge largely overlaps with the substructures at the border between the N- and C-lobes that we predict to be most prone to unfolding. Our model is thus supported by and further corroborates EM-based observations. If the Hsp90 dependency of a kinase is linked to the tendency of the N-lobe and C-lobe to separate in an open state, the interaction surfaces are not random, but should correspond to energetically uncoupled regions that support the conformational reorganization leading to the separation of the two domains. Furthermore, existing data on GR point to regions that are reshaped during the formation of chaperone complexes and that significantly overlap with our designs.

In embodiments the present disclosure provides for selective chemical targeting of Hsp90 PPIs without significant inhibition or degradation of all chaperone clients. The notion that selective clearance of oncogenic clients can be achieved through perturbation of Hsp90 PPIs has implications for both mechanistic studies and for future therapeutic applications. Mechanistically, selective degradation of specific oncoproteins can be used as a complement to molecular biology to investigate their relevance in signaling pathways. In summary, amino-acid stretches with minimal intra-client coupling can be the drivers of selective degradation of specific Hsp90 client proteins. Synthetic mimics of these regions have advantages over ATP-competitive Hsp90 inhibitors. The latter compounds unselectively target the entire spectrum of Hsp90 client proteins, causing their destabilization. This lack of selectivity is frequently associated to toxicity, which limits the clinical application of those agents. In contrast, the perturbation of the Hsp90 PPIs with specific clients by our molecules enables a controlled modulation of chaperone networks.

While the foregoing is directed to embodiments of the present disclosure, other and further embodiments of the disclosure may be devised without departing from the basic scope thereof. It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

What is claimed is:

1. A method of identifying an agent-of-interest that alters binding or activity of a client protein to a chaperone, co-chaperone, or chaperone-co-chaperone complex, the method comprising:
determining a three-dimensional (3D) structure of a client protein-of-interest;
evaluating the 3D structure of the client protein-of-interest to identify an unstable substructure of the 3D structure of the client protein-of-interest; and
determining an amino acid sequence of the unstable substructure of the 3D structure of the client protein-of-interest to identify an agent-of-interest that alters binding or activity of a client protein to a chaperone, co-chaperone, or chaperone-co-chaperone complex.

2. The method of claim 1, further comprising synthesizing a selective peptide inhibitor having an amino acid sequence having at least 90% sequence identity to the amino acid sequence of the unstable substructure of the 3D structure of the client protein-of-interest.

3. The method of claim 2, wherein the selective peptide inhibitor blocks protein interactions or alters binding or activity of a client protein to a chaperone, co-chaperone, or chaperone-co-chaperone complex, and/or induces apoptosis in cancer cells.

4. The method of claim 1, wherein evaluating the 3D structure of the client protein-of-interest to identify an unstable substructure of the 3D structure of the client protein-of-interest further comprises predicting the unstable substructure of the 3D structure of the client protein-of-interest based on energy decomposition.

5. The method of claim 1, wherein the client protein comprises one or more proteins characterized as a heat shock protein (HSP).

6. The method of claim 1, wherein the client protein comprises one or more Hsp90 client proteins.

7. The method of claim 1, wherein the client protein comprises one or more proteins selected from a class comprising a steroid hormone, a receptor, a kinase, a non-signal transduction, a telomerase, or a CFTR.

8. The method of claim 1, wherein the client protein is one or more of c-Abl, c-Src, Cdk4, B-Raf or glucocorticoid receptor.

9. The method of claim 1, wherein the chaperone is an Hsp90 chaperone.

10. The method of claim 1, wherein the co-chaperone is an Hsp90 co-chaperone.

11. The method of claim 1, wherein the co-chaperone is one of CDC37, or Aha1.

12. The method of claim 1, wherein the chaperone-co-chaperone complex is an Hsp90 chaperone-co-chaperone complex.

13. The method of claim 1, wherein the unstable substructure of the 3D structure of the client protein-of-interest is further characterized as an epitope.

14. The method of claim 1, wherein the activity of the client protein to the chaperone-co-chaperone complex is kinase activity and stability.

15. The method of claim 1, wherein the agent-of-interest alter, inhibit, decrease, or destroy one or more cancer cells.

16. The method of claim 1, wherein the agent-of-interest are characterized as permeable to a cell membrane.

17. The method of claim 1, further comprising assaying one or more agents-of-interest for altering post-translational medication of any chaperone, co-chaperone, or client protein.

18. A method of inhibiting, treating, or preventing cancer or metastatic cancer in a subject, the method comprising,
administering a therapeutically effective amount of an agent-of-interest identified by the method of claim 1, or a pharmaceutically acceptable salt or a derivative thereof, to a subject in need of treatment.

19. A method of identifying an agent-of-interest that alters binding or activity of an Hsp90 client protein to an Hsp90 chaperone, Hsp90 co-chaperone, or Hsp90 chaperone-co-chaperone complex, the method comprising:
evaluating a 3D structure of an Hsp90 client protein-of-interest to identify an unstable substructure of the 3D structure of the Hsp90 client protein-of-interest; and
determining an amino acid sequence of the unstable substructure of the 3D structure of the Hsp90 client protein-of-interest to identify an Hsp90 agent-of-interest that alters binding or activity of an Hsp90 client protein to an Hsp90 chaperone, Hsp90 co-chaperone, or Hsp90 chaperone-co-chaperone complex.

20. A synthetic selective peptide inhibitor, comprising:
an amino acid sequence having at least 90% sequence identity to SEQ ID NO: 1, 2, 3, 4, 5, 6, 7, 8, 9, or 10.


SEQUENCE LISTING (10 sequences)

SEQ ID NO: 1 - protein, 31 aa, Artificial Sequence
  Synthetic Sequence
  PEPTIDE (6)..(6): 4-Fluoro-L-phenylalanine
LGGGQFGEVYGGVAVKTLGGGEFLDEAAVMK

SEQ ID NO: 2 - protein, 20 aa, Artificial Sequence
  Synthetic Sequence
  PEPTIDE (1)..(1): 4-Fluoro-L-phenylalanine
FGGSPYPGIDLSQVYELLEK

SEQ ID NO: 3 - protein, 27 aa, Artificaial Sequence
  peptide (22)..(22): 4-Fluoro-L-phenylalanine
GYSTKPQLAGGGNVTAPTPQGFQHSGS

SEQ ID NO: 4 - protein, 32 aa, Artificial Sequence
  Synthetic Sequence
  peptide (14)..(14): 4-Fluoro-L-phenylalanine
FGTVYKGKWGGGGFSTKPQLAGGGNVTAPTPQ

SEQ ID NO: 5 - protein, 35 aa, Artificial Sequence
  Synthetic sequece
  peptide (27)..(27): 4-Fluoro-L-phenylalanine
CATSRTDREGPNGGGGGGGLPISTGGFQMALTPVV

SEQ ID NO: 6 - protein, 27 aa, Artificial Sequence
  Synthetic sequence
  peptide (19)..(19): 4-Fluoro-L-phenylalanine
PVAEIGVGAYGGGRVPGGFQMALTPVV

SEQ ID NO: 7 - protein, 32 aa, Artificial Sequence
  Synthetic Sequence
  peptide (6)..(6): 4-Fluoro-L-phenylalanine
LGQGCFGGKPGTMSPGGEEPGGRESLGWNGTT

SEQ ID NO: 8 - protein, 31 aa, Artificial Sequence
  Synthetic Sequence
  peptide (29)..(29): 4-Fluoro-L-phenylalanine
GEMGKGGKGRVPYPGMVNREVLDQVERGFRM

SEQ ID NO: 9 - protein, 21 aa, Artificial Sequence
  Synthetic sequence
  peptide (16)..(16): 4-Fluoro-L-phenylalanine
TLPCGGTWRIMTGIEFPEMLA

SEQ ID NO: 10 - protein, 29 aa, Artificial Sequence
  synthetic sequence
  peptide (24)..(24): 4-Fluoro-L-phenylalanine
YAGYDSSVPDSTWRIMTTLNMGGFPEMLA